(19) United States
(12) Patent Application Publication
(10) Pub. No.: US 2026/0369421 A1
(43) Pub. Date: Nov. 19, 2026

(54) MACHINE LEARNING DETECTION OF INTRAVENOUS FLUID CONTAMINATION IN BLOOD COUNT RESULTS

(71) Applicant: UNIVERSITY OF UTAH RESEARCH FOUNDATION, Salt Lake City, UT (US)

(72) Inventors: Nicholas Spies, Salt Lake City, UT (US); Carly Maucione, St. Louis, MO (US)

(21) Appl. No.: 19/673,351

(22) Filed: May 11, 2026

Related U.S. Application Data
(60) Provisional application No. 63/804,361, filed on May 12, 2025.

(51) Int. Cl. G16H 50/30; G06N 20/20; G16H 10/40; G16H 10/60; G16H 15/00

(52) U.S. Cl. CPC G16H 50/30; G06N 20/20; G16H 10/40; G16H 10/60; G16H 15/00

(57) ABSTRACT

Described herein is a method for identifying potentially unnecessary red blood cell transfusions includes receiving transfusion data comprising transfusion events, each associated with a complete blood count trio comprising a prior sample collected before a current sample, the current sample collected before the transfusion event, and a post sample collected after the transfusion event. The method includes applying a machine learning model to each trio to generate a contamination prediction indicating a likelihood that the current sample is contaminated by intravenous fluid. For each transfusion event associated with a trio predicted as contaminated, the method includes evaluating whether a post-transfusion hemoglobin concentration exceeds a threshold hemoglobin value and exceeds a prior hemoglobin concentration. The method includes classifying a transfusion event as potentially unnecessary when the trio is predicted as contaminated, the post-transfusion hemoglobin concentration exceeds the threshold value, and the post-transfusion hemoglobin concentration exceeds the prior hemoglobin concentration.

### CROSS-REFERENCE TO RELATED APPLICATIONS

This application claims priority to U.S. Provisional Patent Application No. 63/804,361 filed on May 12, 2025, which is incorporated by reference herein in its entirety.

### BACKGROUND

Complete blood counts (CBCs) are among the most frequently ordered laboratory tests in clinical medicine, providing measurements of hemoglobin concentration, platelet counts, white blood cell counts, and other hematologic parameters. These measurements inform a wide range of clinical decisions, including the administration of red blood cell (RBC) transfusions for patients with anemia.

Specimens collected directly from, or downstream of, an intravenous (IV) catheter can demonstrate substantial measurement error. This phenomenon, referred to as IV fluid contamination (or dilution), occurs when IV fluids mix with the blood specimen, causing dilution of the measured analytes. IV fluid contamination causes operational burdens in the laboratory and disruptions in clinical workflows when detected by routine quality safeguards. However, when such errors go undetected, there is a risk of preventable patient harm due to inappropriate clinical decisions motivated by inaccurate results.

IV fluid contamination often causes an anomaly-with-resolution pattern across all measured analytes. For a contaminated CBC, count and concentration results may initially decrease due to dilution, then rebound toward the patient's baseline when a subsequent specimen is properly collected. The magnitude of these effects depends upon the contaminating fluid's composition, as well as its relative abundance (e.g., volume and dilution factor) in the specimen.

RBC transfusion is a common intervention for symptomatic acute blood loss anemia, though it is not without risk. Transfusion reactions occur in a portion of RBC transfusions, ranging from asymptomatic alloimmunization to life-threatening anaphylaxis or hemolysis. For hospitals and laboratories, the logistic and operational infrastructure to provide transfusion services is substantial, and each unit confers direct acquisition costs to the laboratory. When a CBC specimen is contaminated by IV fluid, the resulting artificially low hemoglobin measurement may prompt a transfusion that would not otherwise be clinically indicated.

Current protocols for detecting IV fluid contamination in real time have limitations, and there is no gold-standard method for identifying these events retrospectively. This makes objective characterization of the problem difficult and hinders the ability to target quality improvement initiatives or optimize laboratory detection protocols. Manual chart review approaches for identifying contamination events are labor-intensive and prone to intra- and inter-reviewer variation.

What is needed are improved systems and methods for detecting IV fluid contamination in CBC results and identifying potentially unnecessary transfusions.

### SUMMARY

One embodiment described herein is a method for identifying potentially unnecessary red blood cell transfusions, the method comprising: receiving, by one or more electronic processors, transfusion data comprising a plurality of red blood cell transfusion events, each transfusion event associated with a complete blood count trio comprising: a prior sample collected before a current sample; the current sample collected before the transfusion event; and a post sample collected after the transfusion event; applying, by the one or more electronic processors, a machine learning model to each complete blood count trio to generate a contamination prediction indicating a likelihood that the current sample is contaminated by intravenous fluid; for each transfusion event associated with a complete blood count trio predicted as contaminated, evaluating, by the one or more electronic processors, whether: a post-transfusion hemoglobin concentration exceeds a threshold hemoglobin value; and the post-transfusion hemoglobin concentration exceeds a prior hemoglobin concentration; and classifying, by the one or more electronic processors, a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration. In one aspect, the threshold hemoglobin value is 8 g/dL. In another aspect, the machine learning model is trained to identify an anomaly-with-resolution pattern in complete blood count results indicative of intravenous fluid contamination. In another aspect, the method further comprises: aggregating, by the one or more electronic processors, the transfusion events classified as potentially unnecessary by at least one of a clinical context, a transfusion indication, a specimen collector identifier, or a collection unit identifier. In another aspect, the method further comprises: calculating, by the one or more electronic processors, an unnecessary transfusion rate based on a ratio of transfusion events classified as potentially unnecessary to a total number of transfusion events for which a complete blood count trio is available. In another aspect, the machine learning model receives input features comprising: a plurality of analyte values comprising a hemoglobin concentration value, a platelet count value, and a white blood cell count value from each of the current sample, the prior sample, and the post sample; a prior delta for each of the plurality of analyte values comprising a difference between analyte values of the prior sample and the current sample; and a post delta for each of the plurality of analyte values comprising a difference between analyte values of the post sample and the current sample. In another aspect, the method further comprises: performing, by the one or more electronic processors, a principal component analysis on the input features to generate one or more principal components; and providing the one or more principal components as additional input features to the machine learning model. In another aspect, the method further comprises: generating, by the one or more electronic processors, a report identifying collection units or specimen collectors associated with elevated rates of potentially unnecessary transfusions. In another aspect, the prior sample and the post sample are collected within 48 hours of the current sample.

Another embodiment described herein is a system for identifying potentially unnecessary red blood cell transfusions, the system comprising: one or more electronic processors; and a memory storing instructions that, when executed by the one or more electronic processors, cause the system to: receive transfusion data comprising a plurality of red blood cell transfusion events, each transfusion event associated with a complete blood count trio comprising a prior sample collected before a current sample, the current sample collected before the transfusion event, and a post sample collected after the transfusion event; and apply a machine learning model to each complete blood count trio to generate a contamination prediction indicating a likelihood that the current sample is contaminated by intravenous fluid. In one aspect, the instructions further cause the system to: perform a principal component analysis on input features to generate one or more principal components; and provide both raw feature values and the one or more principal components as inputs to the machine learning model. In another aspect, the machine learning model is trained using simulated contamination data generated by applying linear dilution to analyte values in a training set of complete blood count trios. In another aspect, the instructions further cause the system to: estimate a mixture ratio for each complete blood count trio predicted as contaminated, the mixture ratio representing a proportion of contaminating fluid relative to a physiologic specimen. In another aspect, the instructions further cause the system to: match a complete blood count trio predicted as contaminated with a corresponding basic metabolic panel collected within a predetermined time window of the current sample; and estimate a mixture ratio for the corresponding basic metabolic panel to provide orthogonal validation of the contamination prediction. In another aspect, the instructions further cause the system to: generate feature importance values using Shapley additive explanation values to identify contributions of individual features to the contamination prediction. In another aspect, the machine learning model comprises a gradient boosting model tuned using cross-validation.

Another embodiment described herein is a non-transitory computer-readable medium storing instructions that, when executed by one or more electronic processors, cause the one or more electronic processors to: receive a plurality of complete blood count trios, each complete blood count trio comprising a prior sample, a current sample, and a post sample, each sample associated with a hemoglobin concentration value, a platelet count value, and a white blood cell count value; calculate, for each complete blood count trio, delta values representing changes in analyte values between the prior sample and the current sample and between the current sample and the post sample; apply a machine learning model trained to detect an anomaly-with-resolution pattern indicative of intravenous fluid contamination to the plurality of complete blood count trios; and generate, for each complete blood count trio, a contamination prediction indicating a likelihood that the current sample is contaminated by intravenous fluid. In one aspect, the instructions further cause the one or more electronic processors to: perform a principal component analysis on input features comprising the hemoglobin concentration value, the platelet count value, the white blood cell count value, and the delta values to generate one or more principal components; and provide both raw feature values and the one or more principal components as inputs to the machine learning model. In another aspect, the machine learning model is trained using a training set in which contamination is simulated by applying linear dilution to analyte values according to a distribution of mixture ratios. In another aspect, the instructions further cause the one or more electronic processors to: estimate a contamination severity for each complete blood count trio predicted as contaminated, the contamination severity comprising an estimated mixture ratio. In another aspect, the instructions further cause the one or more electronic processors to: identify transfusion events associated with complete blood count trios predicted as contaminated; evaluate whether a post-transfusion hemoglobin concentration exceeds a threshold value and exceeds a prior hemoglobin concentration; and classify a transfusion event as potentially unnecessary based on the evaluation. In another aspect, the instructions further cause the one or more electronic processors to: aggregate transfusion events classified as potentially unnecessary by at least one of a specimen collector identifier, a collection unit identifier, or a clinical context. In another aspect, the machine learning model comprises a gradient boosting model. In another aspect, the instructions further cause the one or more electronic processors to: apply a classification threshold to the contamination prediction to generate a binary classification indicating whether the current sample is contaminated.

Another embodiment described herein is a method for detecting intravenous fluid contamination in complete blood count results, the method comprising: receiving, by one or more electronic processors, a plurality of complete blood count trios, each complete blood count trio comprising: a prior sample associated with a first hemoglobin concentration value, a first platelet count value, and a first white blood cell count value; a current sample associated with a second hemoglobin concentration value, a second platelet count value, and a second white blood cell count value; and a post sample associated with a third hemoglobin concentration value, a third platelet count value, and a third white blood cell count value; calculating, by the one or more electronic processors, for each complete blood count trio: a prior delta comprising a difference between the first hemoglobin concentration value and the second hemoglobin concentration value, a difference between the first platelet count value and the second platelet count value, and a difference between the first white blood cell count value and the second white blood cell count value; and a post delta comprising a difference between the third hemoglobin concentration value and the second hemoglobin concentration value, a difference between the third platelet count value and the second platelet count value, and a difference between the third white blood cell count value and the second white blood cell count value; applying, by the one or more electronic processors, a machine learning model to the plurality of complete blood count trios, wherein the machine learning model is trained to distinguish contaminated samples from non-contaminated samples based on an anomaly-with-resolution pattern; and generating, by the one or more electronic processors, for each complete blood count trio, a contamination prediction indicating a likelihood that the current sample is contaminated by intravenous fluid. In one aspect, the machine learning model is trained using a training set comprising a plurality of reference complete blood count trios, wherein contamination is simulated in a portion of the plurality of reference complete blood count trios by applying a linear dilution to analyte values. In another aspect, the linear dilution is applied according to a distribution of mixture ratios representing a proportion of contaminating fluid relative to a physiologic patient specimen. In another aspect, the method further comprises: performing, by the one or more electronic processors, a principal component analysis on input features comprising the second hemoglobin concentration value, the second platelet count value, the second white blood cell count value, the prior delta, and the post delta to generate one or more principal components; and providing the one or more principal components as additional input features to the machine learning model. In another aspect, the machine learning model receives both raw feature values and the one or more principal components as input features. In another aspect, the machine learning model comprises a gradient boosting model. In another aspect, the method further comprises: applying, by the one or more electronic processors, a classification threshold to the contamination prediction to generate a binary classification indicating whether the current sample is contaminated. In another aspect, the classification threshold is selected to achieve a positive predictive value exceeding a predetermined value. In another aspect, the prior sample and the post sample are collected within 48 hours of the current sample. In another aspect, the method further comprises: estimating, by the one or more electronic processors, a contamination severity for each complete blood count trio predicted as contaminated, wherein the contamination severity comprises an estimated mixture ratio representing a proportion of contaminating fluid in the current sample. In another aspect, the method further comprises: aggregating, by the one or more electronic processors, the contamination predictions by at least one of a specimen collector identifier or a collection unit identifier to generate contamination rate data for quality improvement analysis.

### DESCRIPTION OF THE DRAWINGS

FIG. 1A shows a schematic overview of CBC contamination or dilution caused by IV fluids resulting in a reduced value for hemoglobin, white blood cell count, or platelet count, which could anomalously indicate the need for an unnecessary transfusion. FIG. 1B shows a schematic overview of the machine learning approach. CBC, complete blood count; CV, cross-validation; HGB, hemoglobin; PCA, principal component analysis; PLT, platelet count; WBC, white blood cell count. FIG. 1C shows a schematic of the data flow of the machine learning approach.

FIG. 2 shows how the simulation was validated. NS, normal saline; D5-NS, 5% dextrose in normal saline; WBC, white blood cells; RBC, red blood cells; HGB, hemoglobin; HCT, hematocrit; MCV, mean corpuscular volume; PLT, platelets. Units: WBC, K/cumm; RBC, M/cumm; HGB, g/dL; HCT, %; MCV, fl; PLT, K/cumm.

FIG. 3 shows a distribution of mixture ratios for simulated training.

FIG. 4A-B show an ROC curve (FIG. 4A) and a PR curve (FIG. 4B) illustrating performance summary on simulated data in cross-validation. Model performance was evaluated using three individual feature sets in order to assess contributions of PCA. These three feature sets included raw features alone, PCA alone, and combined features plus PCA. PCA was performed on all input and added as engineered features in the PCA alone and Features+PCA sets. Model performance was then assessed by comparing results to the validation sets, and the set with the best auPR, Features+PCA, was selected for use.

FIG. 5 shows a feature set performance evaluation.

FIG. 6A-E show an ROC curve (FIG. 6A) and a PR curve (FIG. 6E) illustrating performance evaluation in a set of 1000 expert-labeled CBC trios. Receiver operating characteristic (ROC) curves and Precision-Recall (PR) curves demonstrate the trade-off in labeling performance across classification thresholds, with the area underneath them (auROC and auPR) summarizing total discriminatory potential. The inset element shows the confusion matrix for each set at a classification threshold of 0.75.

FIG. 7A-E show an ROC curve (FIG. 7A) and a PR curve (FIG. 7E) illustrating performance evaluation in a set of 1000 expert-labeled CBC trios with a RBC transfusion administered between the current and post-CBC results. Receiver operating characteristic (ROC) curves and precision-recall (PR) curves demonstrate the trade-off in labeling performance across classification thresholds, with the area underneath them (auROC and auPR) summarizing total discriminatory potential. The inset element shows the confusion matrix for expert adjudication of contamination.

FIG. 8 shows hemoglobin concentration, platelet count, and WBC count across the three time points (prior, current, post) for the validation set of CBC trios drawn in 2024. CBCs predicted as not contaminated are highlighted in gray (left), while those predicted as contaminated demonstrate the anomaly-with-resolution pattern and are highlighted in dark grey (right). Each boxplot represents the median and interquartile range, with lines extending across the middle 95th percentile. The dotted line across the hemoglobin plot indicates a hemoglobin concentration of 7 g/dL. Table 6 shows hemoglobin, hematocrit, WBC count, and platelet count from the 2024 validation set are summarized in order to compare Contaminated and Not Contaminated specimens at each institution. “Hours to Post” represents the amount of time between the current and post samples, which is notably shorter for predicted contaminated samples at both institutions.

FIG. 9A shows hemoglobin concentration across the three time points (prior, current, post) for the CBC trios drawn in 2024 with justified transfusions (gray, left) and those with potentially unnecessary transfusions (red, right) at each institution. Each boxplot represents the median and interquartile range, with lines extending across the middle 95^{th} percentile. The dotted line indicates the most common hemoglobin threshold for RBC transfusion of 7 g/dL. FIG. 9E shows odds ratios for factors contributing to the predicted necessity of transfusion (odds ratios above 1.00 suggest a higher likelihood that any given transfusion was labeled potentially unnecessary, as compared to the most frequent label for each feature (patients in intensive care units and transfusions with an indication of “hemoglobin <7 g/dL”). Table 7 shows the change in hemoglobin concentration from current to post (g/dL), clinical context, and indication for transfusion for those cases with justified vs. potentially unnecessary transfusions.

FIG. 10 shows estimated contamination severity in the 9767 contaminated real-world CBC trios. Each point represents one predicted contaminated specimen, with those light gray points above the axis representing cases at WashU, and those below the axis at Utah. At Utah, the median mixture ratio was 0.13, with 1669 (76%) above 0.10, and 275 (13%) above 0.20. At WashU, the median mixture ratio was 0.14, with 4995 (66%) above 0.10, and 1046 (14%) above 0.20. The maximum estimated mixture ratio was 0.60 and 0.66 at Utah and WashU, respectively.

FIG. 11 shows mixture ratios in predicted contaminated CBCs compared to the mixture ratios of paired BMPs from WashU. CBCs predicted as contaminated with a concurrently contaminated BMP are shown in each point, with darker points corresponding to higher point density. Correlation between the mixture ratios for the CBCs and BMPs is quantified by the coefficient of determination (R²) and best-fit line (solid red line), while the dashed line represents the identity line (y=x).

FIG. 12A shows a global evaluation of feature importance using Shapley Additive Explanation (SHAP) values, in which features are listed in decreasing order of median magnitude of their contribution. Each point represents one CBC trio in the test set. Points to the right of the center line suggest that value increased the model's predicted probability, while lighter points represent trios where that feature had a higher value. FIG. 12B shows the anomaly-with-resolution pattern as a function of principal component (PC) tertiles, top. Bottom, PC1 and 2 for each CBC trio, colored by change from prior and change to post for hemoglobin (Hgb), platelet count (Plt), and white blood cell count (WBC). Red points represent increases, while blue points represent decreases.

FIG. 13 shows a graph of unnecessary transfusion rates by classification threshold.

FIG. 14 shows a plot of the cumulative impact of line draws by collector.

FIG. 15 shows a plot of the monthly contamination rate by phlebotomist.

FIG. 16 shows a graph of contamination rate by collection unit.

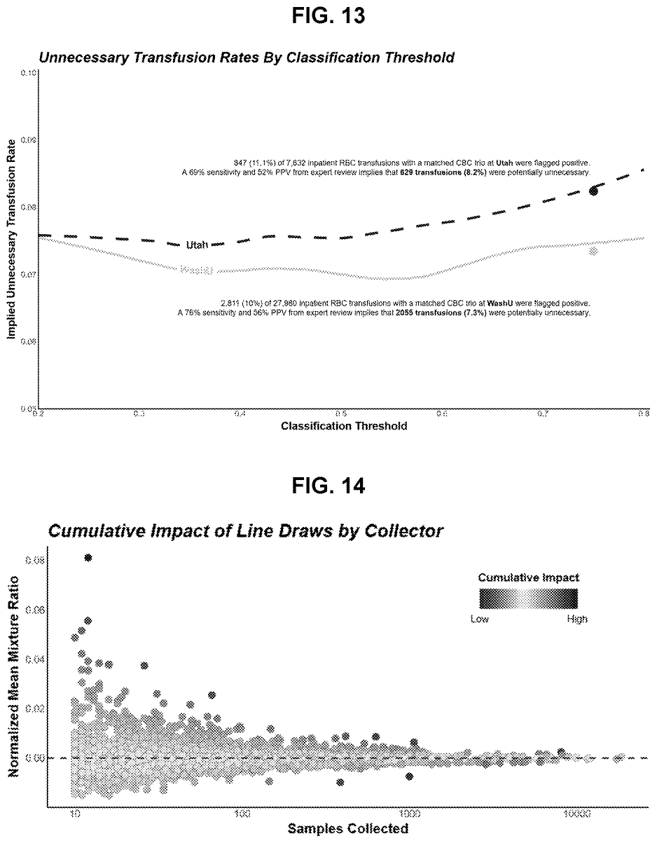

FIG. 17 shows a graph of estimated unnecessary transfusion rate by collecting unit.

FIG. 18 shows a flowchart of a method for identifying potentially unnecessary red blood cell transfusions, according to aspects of the present disclosure.

FIG. 19 shows a flowchart of a method for detecting intravenous fluid contamination in complete blood count results, according to aspects of the present disclosure.

FIG. 20 shows a system diagram for identifying potentially unnecessary red blood cell transfusions, according to aspects of the present disclosure.

### DETAILED DESCRIPTION

Unless otherwise defined, all technical and scientific terms used herein have the same meaning as commonly understood by one of ordinary skill in the art. For example, any nomenclatures used in connection with, and techniques of medicine, clinical chemistry, biochemistry, and chemistry described herein are well known and commonly used in the art. In case of conflict, the present disclosure, including definitions, will control the interpretation of terms. Exemplary methods and materials are described below, although methods and materials similar or equivalent to those described herein can be used in practice or testing of the embodiments and aspects described herein.

As used herein, terms such as “include,” “including,” “contain,” “containing,” “having,” and the like mean “comprising.” The present disclosure also contemplates other embodiments “comprising,” “consisting essentially of,” and “consisting of” the embodiments or elements presented herein, whether explicitly set forth or not. As used herein, “comprising,” is an “open-ended” term that does not exclude additional, unrecited elements or method steps. As used herein, “consisting essentially of” limits the scope of a claim to the specified materials or steps and those that do not materially affect the basic and novel characteristics of the claimed invention. As used herein, “consisting of” excludes any element, step, or ingredient not specified in the claim.

As used herein, the term “a,” “an,” “the” and similar terms used in the context of the disclosure (especially in the context of the claims) are to be construed to cover both the singular and plural unless otherwise indicated herein or clearly contradicted by the context. In addition, “a,” “an,” or “the” means “one or more” unless otherwise specified.

As used herein, the term “or” can be conjunctive or disjunctive.

As used herein, the term “and/or” refers to both the conjunctive and disjunctive.

As used herein, the term “substantially” means to a great or significant extent, but not completely.

As used herein, the term “about” or “approximately” as applied to one or more values of interest, refers to a value that is similar to a stated reference value, or within an acceptable error range for the particular value as determined by one of ordinary skill in the art, which will depend in part on how the value is measured or determined, such as the limitations of the measurement system. In one aspect, the term “about” refers to any values, including both integers and fractional components that are within a variation of up to ±10% of the value modified by the term “about.” Alternatively, “about” can mean within 3 or more standard deviations, per the practice in the art. Alternatively, such as with respect to biological systems or processes, the term “about” can mean within an order of magnitude, in some embodiments within 5-fold, and in some embodiments within 2-fold, of a value. As used herein, the symbol “~” means “about” or “approximately.”

All ranges disclosed herein include both end points as discrete values as well as all integers and fractions specified within the range. For example, a range of 0.1-2.0 includes 0.1, 0.2, 0.3, 0.4 . . . 2.0. If the end points are modified by the term “about,” the range specified is expanded by a variation of up to ±10% of any value within the range or within 3 or more standard deviations, including the end points, or as described above in the definition of “about.”

For ease of description, some or all of the example systems presented herein may be described with a single exemplar of each of its component parts. Some examples may not describe or illustrate all components of the systems. Other examples may include more or fewer of each of the illustrated components, may combine some components, or may include additional or alternative components.

Unless the context of their usage unambiguously indicates otherwise, the articles “a,” “an,” and “the” should not be interpreted as meaning “one” or “only one.” Rather these articles should be interpreted as meaning “at least one” or “one or more.” Likewise, when the terms “the” or “said” are used to refer to a noun previously introduced by the indefinite article “a” or “an,” “the” and “said” mean “at least one” or “one or more” unless the usage unambiguously indicates otherwise.

It should also be understood that although certain drawings illustrate hardware and software located within particular devices and systems, these depictions are for illustrative purposes only. In some implementations, the illustrated components may be combined or divided into separate software, firmware and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

Thus, in the claims, if an apparatus or system is claimed, for example, as including an electronic processor or other element configured in a certain manner, for example, to make multiple determinations, the claim or claim element should be interpreted as meaning one or more electronic processors (or other element) where any one of the one or more electronic processors (or other element) is configured (programmed or specifically designed) as claimed, for example, to make some or all of the multiple determinations. To reiterate, those electronic processors and processing may be distributed.

As used herein, the terms “room temperature,” “RT,” or “ambient temperature” refer to the typical temperature in an indoor laboratory setting. In one aspect, the laboratory setting is climate controlled to maintain the temperature at a substantially uniform temperature or with a specific range of temperatures. In one aspect, “room temperature” refers a temperature of about 15-30 °C, including all integers and endpoints within the specified range. In another aspect, “room temperature” refers a temperature of about 15-30 °C; about 20-30 °C; about 22-30 °C; about 25-30 °C; about 27-30 °C; about 15-22 °C; about 15-25 °C; about 15-27 °C; about 20-22 °C; about 20-25 °C; about 20-27 °C; about 22-25 °C; about 22-27 °C; about 25-27 °C; about 15 °C±10%; about 20 °C±10%; about 22 °C±10%; about 25 °C±10%; about 27 °C±10%; ~20 °C, ~22 °C, ~25 °C, or ~27 °C, at standard atmospheric pressure.

As used herein, the terms “control,” or “reference” are used herein interchangeably. A “reference” or “control” level may be a predetermined value or range, which is employed as a baseline or benchmark against which to assess a measured result. “Control” also refers to control experiments.

As used herein, the terms “amino acid,” “nucleotide,” “polynucleotide,” “vector,” “polypeptide,” and “protein” have their common meanings as would be understood by a biochemist of ordinary skill in the art. Standard single letter nucleotides (A, C, G, T, U) and standard single letter amino acids (A, C, D, E, F, G, H, I, K, L, M, N, P, Q, R, S, T, V, W, or Y) are used herein.

As used herein, the terms “active ingredient” or “active pharmaceutical ingredient” refer to a pharmaceutical agent, active ingredient, compound, or substance, compositions, or mixtures thereof, that provide a pharmacological, often beneficial, effect.

As used herein, the term “dose” denotes any form of an active ingredient formulation or composition, including cells, that contains an amount sufficient to initiate or produce a therapeutic effect with at least one or more administrations. “Formulation” and “composition” are used interchangeably herein.

As used herein, the term “prophylaxis” refers to preventing or reducing the progression of a disorder, either to a statistically significant degree or to a degree detectable by a person of ordinary skill in the art.

As used herein, the terms “effective amount” or “therapeutically effective amount,” refers to a substantially non-toxic, but sufficient amount of an action, agent, composition, or cell(s) being administered to a subject that will prevent, treat, or ameliorate to some extent one or more of the symptoms of the disease or condition being experienced or that the subject is susceptible to contracting. The result can be the reduction or alleviation of the signs, symptoms, or causes of a disease, or any other desired alteration of a biological system. An effective amount may be based on factors individual to each subject, including, but not limited to, the subject's age, size, type or extent of disease, stage of the disease, route of administration, the type or extent of supplemental therapy used, ongoing disease process, and type of treatment desired.

As used herein, the term “subject” refers to an animal. Typically, the subject is a mammal. A subject also refers to primates (e.g., humans, male or female; infant, adolescent, or adult), non-human primates, rats, mice, rabbits, pigs, cows, sheep, goats, horses, dogs, cats, fish, birds, and the like. In one embodiment, the subject is a primate. In one embodiment, the subject is a human.

As used herein, a subject is “in need of treatment” if such subject would benefit biologically, medically, or in quality of life from such treatment. A subject in need of treatment does not necessarily present symptoms, particular in the case of preventative or prophylaxis treatments.

As used herein, the terms “inhibit,” “inhibition,” or “inhibiting” refer to the reduction or suppression of a given biological process, condition, symptom, disorder, or disease, or a significant decrease in the baseline activity of a biological activity or process.

As used herein, “treatment” or “treating” refers to prophylaxis of, preventing, suppressing, repressing, reversing, alleviating, ameliorating, or inhibiting the progress of biological process including a disorder or disease, or completely eliminating a disease. A treatment may be either performed in an acute or chronic way. The term “treatment” also refers to reducing the severity of a disease or symptoms associated with such disease prior to affliction with the disease. “Repressing” or “ameliorating” a disease, disorder, or the symptoms thereof involves administering a cell, composition, or compound described herein to a subject after clinical appearance of such disease, disorder, or its symptoms. “Prophylaxis of” or “preventing” a disease, disorder, or the symptoms thereof involves administering a cell, composition, or compound described herein to a subject prior to onset of the disease, disorder, or the symptoms thereof. “Suppressing” a disease or disorder involves administering a cell, composition, or compound described herein to a subject after induction of the disease or disorder thereof but before its clinical appearance or symptoms thereof have manifest.

As used herein, the terms “complete blood count” or “CBC” refer to a laboratory test that measures various components of blood, including hemoglobin concentration, platelet count, white blood cell count, hematocrit, and other hematologic parameters. A CBC may be performed on a blood specimen collected from a patient and analyzed using an automated hematology system.

As used herein, the term “CBC trio” or “complete blood count trio” refers to a set of three complete blood count results from a single patient comprising a prior sample, a current sample, and a post sample. The prior sample is collected before the current sample, and the post sample is collected after the current sample. In some cases, the prior sample and the post sample are collected within 48 hours of the current sample.

As used herein, the term “contamination” refers to the dilution of a blood draw sample from a subject that is or may be diluted by intravenous fluid administration, such as from an IV line, flushes, IV drug administration, or other intravenous fluid administration temporally proximate to the blood sample acquisition. Actual fluid contamination or dilution of a blood sample can result in anomalous complete blood count values, where the values are observed as being “lower” relative to the prior sample and/or post sample from the same subject. These anomalously lower values may incorrectly indicate to a clinician that a transfusion or other intervention is required, when none is actually required. The anomalously low CBC values of the contaminated sample are due to the dilution of blood by the intravenous fluid administration temporally proximate to the blood sample acquisition.

As used herein, the term “contamination prediction” refers to an output generated by a machine learning model indicating a likelihood that a blood specimen, such as a current sample in a CBC trio, is contaminated by intravenous fluid. The contamination prediction may be expressed as a probability value between 0 and 1, where higher values indicate a greater likelihood of contamination. In some cases, a classification threshold may be applied to the contamination prediction to generate a binary classification indicating whether the sample is contaminated.

As used herein, the term “anomaly-with-resolution pattern” refers to a characteristic pattern observed in sequential laboratory results indicative of intravenous fluid contamination. In the anomaly-with-resolution pattern, count and concentration results in a contaminated specimen initially decrease due to dilution, then rebound toward the patient's baseline when a subsequent specimen is properly collected. The anomaly-with-resolution pattern may be observed across multiple analytes, including hemoglobin concentration, platelet count, and white blood cell count.

As used herein, the term “mixture ratio” refers to a proportion of contaminating fluid relative to a physiologic patient specimen (e.g., a blood sample). The mixture ratio may be expressed as a value between 0 and 1, where a mixture ratio of 0 indicates no contamination and higher values indicate greater proportions of contaminating fluid. In some cases, the mixture ratio may be estimated using regression models trained on simulated contamination data.

As used herein, the term “hemoglobin concentration” refers to a measurement of the amount of hemoglobin protein present in a blood specimen, typically expressed in grams per deciliter (g/dL). Hemoglobin concentration may be measured as part of a complete blood count and may be used to assess anemia and inform transfusion decisions.

As used herein, the term “platelet count” refers to a measurement of the number of platelets present in a blood specimen, typically expressed in thousands per cubic millimeter (K/cumm). Platelet count may be measured as part of a complete blood count.

As used herein, the term “white blood cell count” or “WBC count” refers to a measurement of the number of white blood cells present in a blood specimen, typically expressed in thousands per cubic millimeter (K/cumm). White blood cell count may be measured as part of a complete blood count.

As used herein, the term “prior delta” refers to a set of differences between analyte values of a prior sample and a current sample in a CBC trio. The prior delta may comprise a difference between a hemoglobin concentration value of the prior sample and a hemoglobin concentration value of the current sample, a difference between a platelet count value of the prior sample and a platelet count value of the current sample, and a difference between a white blood cell count value of the prior sample and a white blood cell count value of the current sample.

As used herein, the term “post delta” refers to a set of differences between analyte values of a post sample and a current sample in a CBC trio. The post delta may comprise a difference between a hemoglobin concentration value of the post sample and a hemoglobin concentration value of the current sample, a difference between a platelet count value of the post sample and a platelet count value of the current sample, and a difference between a white blood cell count value of the post sample and a white blood cell count value of the current sample.

As used herein, the term “threshold hemoglobin value” refers to a hemoglobin concentration value used to evaluate whether a transfusion event may be potentially unnecessary. In some cases, the threshold hemoglobin value may be 8 g/dL.

As used herein, the term “principal component analysis” or “PCA” refers to a dimensionality reduction technique that transforms a set of input features into a set of linearly uncorrelated variables called principal components. Principal component analysis may be used to capture multi-dimensional patterns, such as the anomaly-with-resolution pattern, in a dimensionally reduced space. In some cases, principal components may be provided as additional input features to a machine learning model along with raw feature values.

As used herein, the term “gradient boosting model” refers to a type of machine learning model that combines multiple weak prediction models, typically decision trees, in an iterative fashion to produce a stronger predictive model. In some cases, the gradient boosting model may be implemented using LightGBM or similar frameworks. The gradient boosting model may be tuned using cross-validation to optimize performance metrics such as log-loss.

As used herein, the term “SHAP values” or “Shapley additive explanation values” refers to a method for explaining individual predictions of a machine learning model by assigning an importance value to each feature's contribution to the model's output. SHAP values may be used to provide both local explanations for individual predictions and global explanations aggregated across multiple predictions to identify which features exert the strongest influence on model outputs.

As used herein, the term “transfusion event” refers to an administration of a blood product, such as a red blood cell unit, to a patient. A transfusion event may be associated with a CBC trio comprising a prior sample collected before a current sample, the current sample collected before the transfusion event, and a post sample collected after the transfusion event.

As used herein, the term “potentially unnecessary transfusion” refers to a transfusion event that may have been motivated by dilutional effects of intravenous fluid contamination rather than by clinically indicated factors. In some cases, a transfusion event may be classified as potentially unnecessary when the CBC trio associated with the transfusion event is predicted as contaminated, a post-transfusion hemoglobin concentration exceeds a threshold hemoglobin value, and the post-transfusion hemoglobin concentration exceeds a prior hemoglobin concentration.

### Overview

The present disclosure relates to systems and methods for detecting intravenous (IV) fluid contamination in complete blood count (CBC) results and for identifying red blood cell (RBC) transfusions that may be unnecessary due to contamination-induced measurement errors.

A complete blood count is performed by obtaining a sample of blood from a subject by venipuncture or from an intravenous port, intravenous catheter, or intravenous lock. The blood sample is typically analyzed using an automated hematology analyzer to measure red blood cells, white blood cells, platelets, hemoglobin (Hgb) levels, hematocrit (Hct) levels, and potentially a differential of the white blood cells (e.g., differentiating the leukocytes into neutrophils, lymphocytes, monocytes, eosinophils, and basophils).

Blood specimens collected directly from, or downstream of, an intravenous catheter may demonstrate substantial measurement error when IV fluids mix with the blood specimen. This phenomenon, referred to as IV fluid contamination, causes dilution of measured analytes in the specimen. When IV fluid contamination occurs, count and concentration results in the contaminated specimen may initially decrease due to dilution, then rebound toward the patient's baseline when a subsequent specimen is properly collected. This characteristic pattern, referred to herein as an anomaly-with-resolution pattern, may be observed across multiple analytes measured in a CBC, including hemoglobin concentration, platelet count, and white blood cell count.

IV fluid contamination may cause operational burdens in the laboratory and disruptions in clinical workflows when detected by routine quality safeguards. However, when such errors go undetected, there is a risk of preventable patient harm due to inappropriate clinical decisions motivated by inaccurate results. In some cases, an artificially low hemoglobin measurement resulting from IV fluid contamination may prompt a transfusion that would not otherwise be clinically indicated.

RBC transfusion is a common intervention for symptomatic acute blood loss anemia, though transfusion is not without risk. Transfusion reactions may occur in a portion of RBC transfusions, ranging from asymptomatic alloimmunization to life-threatening anaphylaxis or hemolysis. For hospitals and laboratories, the logistic and operational infrastructure to provide transfusion services is substantial, and each unit confers direct acquisition costs to the laboratory.

The methods and systems described herein may utilize machine learning models to identify IV fluid contamination in CBC results by detecting the anomaly-with-resolution pattern across sequential laboratory measurements. In some cases, a CBC trio comprising a prior sample, a current sample, and a post sample may be analyzed to determine whether the current sample exhibits characteristics indicative of IV fluid contamination. The machine learning models may be trained using simulated contamination data generated by applying linear dilution to analyte values in a training set of CBC trios.

In some aspects, the methods and systems may further evaluate whether a transfusion event associated with a contaminated CBC may be potentially unnecessary. A transfusion event may be classified as potentially unnecessary when the CBC trio associated with the transfusion event is predicted as contaminated, a post-transfusion hemoglobin concentration exceeds a threshold hemoglobin value, and the post-transfusion hemoglobin concentration exceeds a prior hemoglobin concentration. In some cases, the threshold hemoglobin value may be 8 g/dL. This value was defined using appropriate clinical context and best-practice guidelines. See e.g., Carson et al., J. Am. Med. Assoc. 330(19): 1892-1902 (2023), which is incorporated by reference herein.

The methods and systems described herein may provide mechanisms for aggregating contamination predictions and potentially unnecessary transfusion classifications by specimen collector identifier, collection unit identifier, or clinical context to support quality improvement analysis. In some cases, contamination severity may be estimated using a mixture ratio representing a proportion of contaminating fluid relative to a physiologic patient specimen.

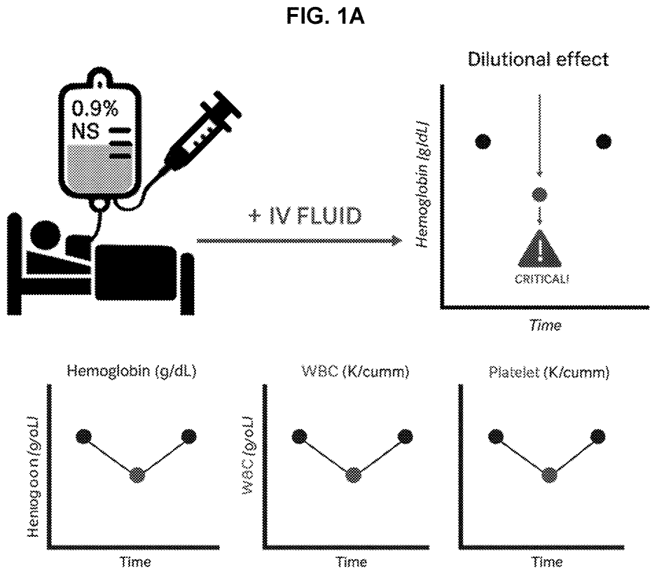

FIG. 1A depicts a schematic overview of complete blood count (CBC) contamination or dilution caused by intravenous (IV) fluids. When a blood specimen is collected directly from, or downstream of, an IV catheter, IV fluids may mix with the blood specimen and cause dilution of measured analytes. IV fluid contamination produces characteristic dilutional effects in CBC results, causing decreased hemoglobin concentration, platelet count, and white blood cell count values. The contaminating IV fluids may include normal saline (NS) and 5% dextrose in normal saline (D5-NS) as specific types of contaminating fluids.

### IV Fluid Contamination and Anomaly-with-Resolution Pattern

As shown in FIG. 1A, IV fluid contamination may produce an anomaly-with-resolution pattern across all measured analytes in a CBC. In the anomaly-with-resolution pattern, count and concentration results in a contaminated specimen initially decrease due to dilution from the contaminating fluid. When a subsequent specimen is properly collected without contamination, the measured values rebound toward the patient's baseline. The anomaly-with-resolution pattern may be observed across multiple analytes measured in a CBC, including hemoglobin concentration, platelet count, and white blood cell count. These values are typically obtained from a CBC using an automated hematology analyzer and the data may be stored in a data storage medium.

The magnitude of the dilutional effects depends upon the contaminating fluid's composition, as well as the relative abundance of the contaminating fluid in the specimen. The relative abundance of contaminating fluid in the specimen may be expressed as a mixture ratio, where higher mixture ratios indicate greater proportions of contaminating fluid relative to the physiologic patient specimen. For example, a mixture ratio of 0.3 indicates that approximately 30% of the specimen volume comprises contaminating IV fluid.

In some cases, the dilutional effects of IV fluid contamination may cause an artificially low hemoglobin measurement that prompts a transfusion that would not otherwise be clinically indicated. As depicted in FIG. 1A, a contaminated CBC may show a hemoglobin concentration that drops to a level marked as anomalous, followed by a return to approximately the initial value when a subsequent specimen is properly collected. The same anomaly-with-resolution pattern may be observed in platelet count and white blood cell count measurements.

A machine learning model may be trained to identify the anomaly-with-resolution pattern in complete blood count results indicative of intravenous fluid contamination. The machine learning model may analyze a CBC trio comprising a prior sample, a current sample, and a post sample to detect whether the current sample exhibits the characteristic dilutional decrease followed by resolution in the post sample. By detecting the anomaly-with-resolution pattern, the machine learning model may generate a contamination prediction indicating a likelihood that the current sample is contaminated by IV fluid.

FIG. 1B illustrates a machine learning pipeline for detecting contamination in complete blood counts (CBCs). The machine learning pipeline may be developed using R 4.3.2 and the tidymodels ecosystem as a software framework for model development. Machine learning pipelines may be developed independently for each institution to generate site-specific models.

FIG. 1C shows a schematic of the data flow of an exemplary machine learning pipeline described herein. CBC trios (HGB, PLT, WBC, Δ_{Prior}, and Δ_{Post}) are preprocessed and used as input for a machine learning model. Preprocessing steps may include, but are not limited to, dropping rows with missing values, dropping rows without prior and post data within 48 hours of an index CBC, simulating contamination at varying severity in a subset of cases, and performing PCA on all input features. In some implementations, preprocessing steps are identical across sites. In some implementations, preprocessing steps are specific to each site.

In some implementations, a training set for each institution is used separately to train a site-specific model. In some implementations, all CBC trios are used to train a global model. In some implementations, the model is a LightGBM model.

The trained model is used to evaluate CBCs according to a method or operation described herein. In some implementations, three test are performed per site. In a non-limiting exemplary evaluation step shown in FIG. 1C, 1000 randomly selected, unlabeled CBCs and 1000 randomly selected, unlabeled CBCs for which an RBC transfusion was given between current and post, were labeled by expert review.

The output of the model may be further processed in a postprocessing step. The model output may be processed in any suitable manner for processing or reporting data. Exemplary postprocessing steps may include, but are not limited to, preparing a graph of the model output or sorting the model output into subsets for further analysis or reporting.

### Machine Learning Pipeline

The machine learning pipeline receives a plurality of complete blood count trios, where each complete blood count trio comprises a prior sample, a current sample, and a post sample. Each sample is associated with a hemoglobin concentration value, a platelet count value, and a white blood cell count value. Input features for the machine learning pipeline may include the hemoglobin concentration value, the platelet count value, and the white blood cell count value from the current sample, as well as, optionally, delta values representing changes in analyte values between the prior sample and the current sample (Δ_{Prior}) and between the current sample and the post sample (Δ_{Post}).

In a situation where delta values (Δ_{Prior}, and Δ_{Post}) are not provided as inputs, The machine learning pipeline calculates, for each complete blood count trio, delta values representing changes in analyte values between the prior sample and the current sample and between the current sample and the post sample. The delta values may comprise a prior delta representing differences between analyte values of the prior sample and the current sample, and a post delta representing differences between analyte values of the post sample and the current sample. In some examples, the machine learning pipeline uses the following data as input to the model: the current hemoglobin concentration value, the current second platelet count value, the current white blood cell count value, the prior delta, and the post delta. Accordingly, in some examples, the machine learning pipeline receives such data as input as compared to receiving the trio and calculating the deltas.

Preprocessing operations may be performed on the input data prior to model training. In some cases, preprocessing may include dropping rows with missing values and dropping rows without prior and post measurements within a predetermined time window of the index CBC. Preprocessing may further include simulating contamination at varying severity in a portion of the training data. In some cases, contamination may be simulated in 50% of CBC trios in the training set.

The contamination simulation applies linear dilution to analyte values in a random selection of CBC trios in the training set. Contamination severities may be drawn from a distribution of mixture ratios that mimics real-world contamination events. All non-simulated trios may be labeled negative for contamination. The linear dilution simulation may be validated using serial dilutions of real-world patient specimens.

Principal component analysis (PCA) may be performed on the input features to generate one or more principal components. Given that the error-generating process of dilution by contaminating fluid is linear and proportional across all analytes, principal component analysis may effectively capture the resulting anomaly-with-resolution pattern from diluted specimens. However, residual biological and analytical variation may remain after the variance in the dilutional effect is captured by principal components.

Three feature sets may be evaluated during model development: features alone, principal components alone, and a combined approach using both features and principal components. The combined approach that includes both raw feature values and principal components may demonstrate improved performance compared to using either features alone or principal components alone. In some cases, the machine learning model receives both raw feature values and the one or more principal components as input features.

The machine learning model may comprise a gradient boosting model. In some cases, the machine learning model comprises a LightGBM (Light Gradient Boosting Machine) model, which is a gradient boosting framework. The LightGBM model may be tuned to log-loss in 5-fold cross-validation. Short tree depths and high splitting fractions may be used in the model to add robustness against overfitting from the simulated training set. Final models may be fit to full training sets before being applied to independent test sets.

Training sets can comprise CBC trios collected over multiple years. In some cases, training sets may comprise 278,447 trios at a first institution and 945,736 trios at a second institution from 2021 to 2023. Test sets may comprise CBC trios from a subsequent time period. In some cases, test sets may comprise 110,299 trios at the first institution and 322,025 trios at the second institution from 2024.

The machine learning model is trained to detect an anomaly-with-resolution pattern indicative of intravenous fluid contamination. The machine learning model applies to the plurality of complete blood count trios and generates, for each complete blood count trio, a contamination prediction indicating a likelihood that the current sample is contaminated by intravenous fluid.

Evaluation of the machine learning pipeline may be performed using expert-labeled test sets. In some cases, a first test set may comprise 1000 randomly selected CBC trios labeled by expert review to provide an unbiased performance assessment. A second test set may comprise 1000 randomly selected CBC trios for which a red blood cell transfusion was administered between the current and post measurements, also labeled by expert review, to evaluate whether the model's inferences are robust to transfusion-induced hemoglobin increases. A third test set may comprise all CBC trios from a test period without labels for application to real-world data.

A binary classification threshold may be applied to the contamination prediction to generate a binary classification indicating whether the current sample is contaminated. In some cases, the classification threshold may be selected to achieve a positive predictive value exceeding a predetermined value. Performance metrics may be calculated using labels from manual chart review as ground truth.

### Validation of Linear Dilution Simulation

As described above, the machine learning model may be trained using a training set comprising a plurality of reference complete blood count trios, wherein contamination is simulated in a portion of the plurality of reference complete blood count trios by applying a linear dilution to analyte values. The linear dilution may be applied according to a distribution of mixture ratios representing a proportion of contaminating fluid relative to a physiologic patient specimen. To validate the linear dilution simulation, in vitro mixing studies may be performed using real-world patient specimens.

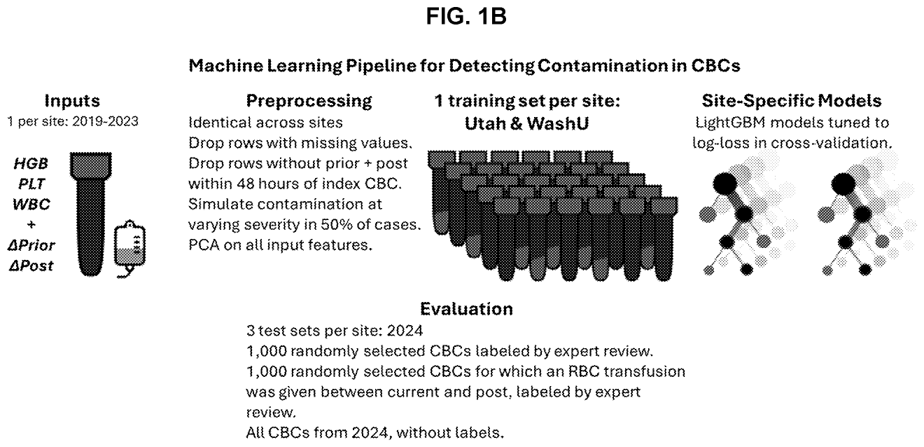

FIG. 2 depicts a graph showing observed values versus expected values by mixture ratio for blood count parameters across two experiments. In the in vitro validation protocol, blood may be drawn using sterile technique into ethylenediaminetetraacetic acid (EDTA) tubes and inverted immediately several times to mix. The blood may then be aliquoted into specimen tubes with intravenous fluid at mixture ratios of 0.1, 0.3, and 0.5. The intravenous fluid may comprise normal saline (NS) or 5% dextrose in normal saline (D5-NS). A total blood volume in each specimen tube, including a control specimen with a mixture ratio of 0, may be 1 mL.

Complete blood count analysis may be performed on a Sysmex XN-Series Automated Hematology System. In some cases, the in vitro samples may be analyzed at 15 minutes after sample preparation. To account for matrix effects and to simulate specimen transport time in a clinical setting, each sample may be run in duplicate at 45 minutes after sample preparation.

Identical experiments using normal saline and 5% dextrose in normal saline may be performed over multiple days using blood from different donors. As shown in FIG. 2, measured values for hematocrit, hemoglobin concentration, platelet count, and white blood cell count may be plotted alongside expected values calculated from the mixture ratios. The measured values demonstrate a linear decrease as the mixture ratio increases from 0.1 to 0.5. The observed values correlate with the expected values calculated from the mixture ratios, with coefficients of determination (R²) of 1.00 for hematocrit, hemoglobin concentration, and platelet count, and 0.99 for white blood cell count. The correlation between measured values and expected values demonstrates that the linear dilution simulation accurately models the dilutional effects of intravenous fluid contamination on complete blood count results.

### Distribution of Mixture Ratios for Training

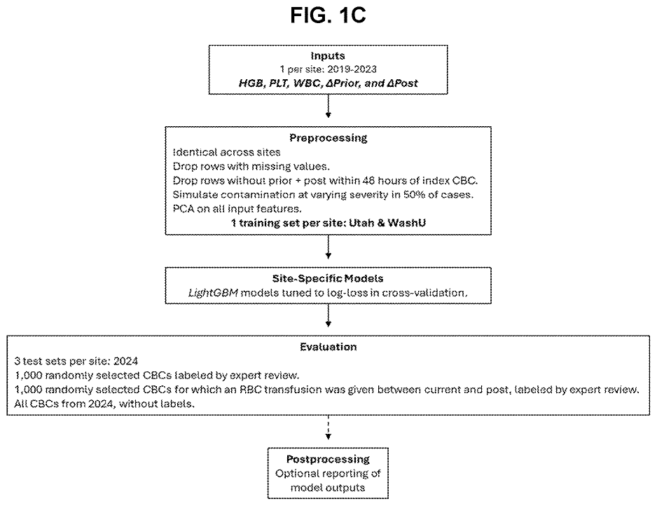

FIG. 3 depicts a graph showing a distribution of mixture ratios for simulated training. As described above, the machine learning model may be trained using a training set in which contamination is simulated by applying linear dilution to analyte values according to a distribution of mixture ratios. The mixture ratio represents a proportion of contaminating fluid relative to a physiologic patient specimen, where a mixture ratio of 0 indicates no contamination and higher values indicate greater proportions of contaminating fluid.

The distribution of mixture ratios used for simulated training may be designed to mimic real-world contamination events. As shown in FIG. 3, the distribution may exhibit a right-skewed pattern in which lower mixture ratios have higher relative frequencies compared to higher mixture ratios. The distribution may rise steeply from lower mixture ratio values, reaching a peak relative frequency at mixture ratios of approximately 0.15 to 0.20. After reaching the peak, the distribution may descend gradually as the mixture ratio increases toward 1.00, with the relative frequency approaching zero at higher mixture ratios.

The shape of the distribution reflects that real-world contamination events may more frequently involve lower proportions of contaminating fluid relative to the physiological patient specimen. In some cases, contamination events with mixture ratios below 0.20 may occur more frequently than contamination events with higher mixture ratios. The distribution may extend from mixture ratios of approximately 0.05 to mixture ratios approaching 1.00, with the majority of simulated contamination events concentrated at lower mixture ratios.

By drawing contamination severities from a distribution that mimics real-world contamination events, the machine learning model may be trained to detect contamination across a range of severities that correspond to contamination patterns observed in clinical practice. In some cases, the distribution may be empirically derived from observations of contamination severity in real-world specimens. The use of a distribution of mixture ratios for simulated training may enable the machine learning model to distinguish contaminated samples from non-contaminated samples across varying levels of contamination severity.

### Model Performance on Simulated Data

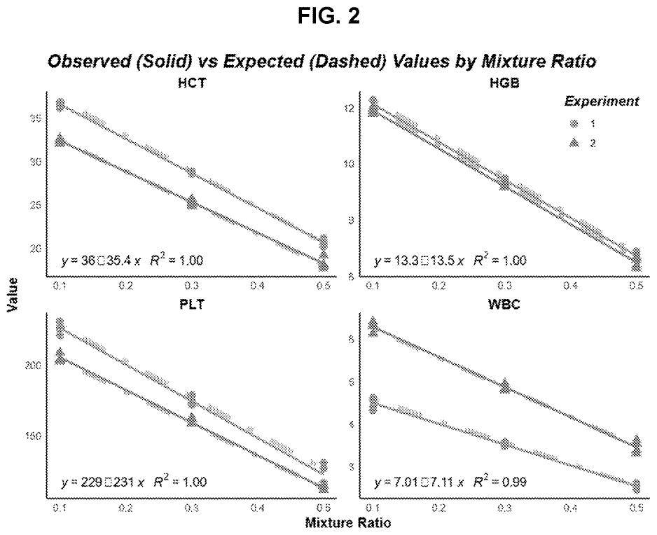

FIG. 4A depicts receiver operating characteristic (ROC) curves and FIG. 4B shows precision-recall (PR) curves for performance evaluation on simulated data in cross-validation. Performance evaluation of the machine learning model on simulated contamination data in the training set may be performed using five-fold cross-validation. The machine learning pipelines may demonstrate capability in distinguishing simulated contamination from real-world results. In some cases, the machine learning model may achieve an area under the receiver operating characteristic curve (auROC) of 0.98 at a first institution and 0.99 at a second institution. The area under the precision-recall curve (auPR) may be 0.99 for both institutions.

As shown in FIG. 4A, the ROC curves for both institutions may demonstrate discriminatory performance, with the curves positioned in the upper left region of the plot. As shown in FIG. 4B, the PR curves may maintain precision across recall values. Performance metrics may include Matthews correlation coefficient (MCC) values of 0.86 at the first institution and 0.88 at the second institution, sensitivity values of 0.88 at the first institution and 0.90 at the second institution, and specificity values of 0.98 for both institutions.

### Feature Set Comparison

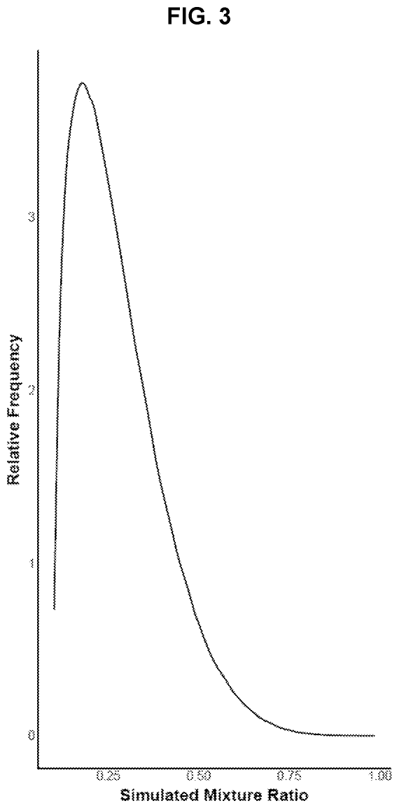

FIG. 5 depicts a bar chart comparing model performance across different feature sets. Model performance may be evaluated using three individual feature sets to assess contributions of principal component analysis (PCA). The three feature sets may include raw features alone, principal components alone, and a combined approach using both features and principal components. PCA may be performed on all input features and added as engineered features in the principal components alone and combined feature sets.

As shown in FIG. 5, the combined approach that includes both raw feature values and principal components may demonstrate improved performance compared to using either features alone or principal components alone. The features alone configuration may achieve an auPR of approximately 0.6. The principal components alone configuration may demonstrate an auPR of approximately 0.62. The combined features plus PCA configuration may achieve an auPR of approximately 0.67.

Model performance may be assessed by comparing results to validation sets, and the feature set with the highest auPR may be selected for use. In some cases, the combined approach using both features and principal components may be selected based on auPR performance. Given that the error-generating process of dilution by contaminating fluid is linear and proportional across all analytes, principal component analysis may effectively capture the resulting anomaly-with-resolution pattern from diluted specimens. However, residual biological and analytical variation may remain after the variance in the dilutional effect is captured by principal components. As such, including both raw feature values and principal components may provide improved performance by capturing both the dilutional pattern and residual variation.

### Real-World Performance Evaluation

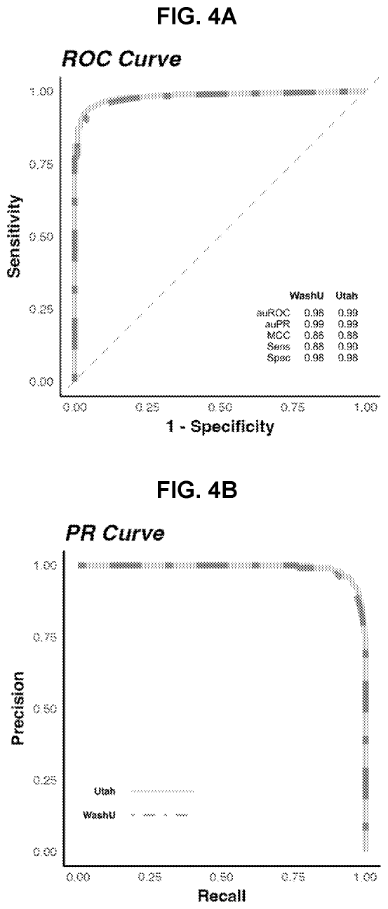

FIG. 6A and FIG. 6B depict receiver operating characteristic (ROC) curves and precision-recall (PR) curves for evaluating machine learning model performance in identifying intravenous fluid contamination across complete blood count (CBC) trios from two institutions. Real-world performance of the machine learning model may be assessed using expert-labeled validation sets comprising CBC trios randomly selected from each institution.

In some cases, validation sets may comprise 1000 randomly selected CBC trios at each institution, with expert review providing ground truth labels for contamination status. The machine learning model may achieve an area under the receiver operating characteristic curve (auROC) of 0.972 at a first institution and 0.957 at a second institution on real-world validation sets. The machine learning model may achieve an area under the precision-recall curve (auPR) of 0.723 at the first institution and 0.619 at the second institution on real-world validation sets. The ROC curves demonstrate discriminatory performance, with the curves positioned in the upper left region of the plot, indicating the ability of the machine learning model to distinguish contaminated samples from non-contaminated samples.

As described above, a classification threshold may be applied to the contamination prediction to generate a binary classification indicating whether the current sample is contaminated. In some cases, the classification threshold may be selected to achieve a positive predictive value exceeding a predetermined value. A classification threshold of 0.75 in the model output may correspond to achieving a positive predictive value of greater than 0.80.

At the classification threshold of 0.75, the machine learning model may achieve a sensitivity of 0.40, a specificity of 0.995, a positive predictive value (PPV) of 0.808, and a negative predictive value (NPV) of 0.967 at the first institution. At the second institution, the machine learning model may achieve a sensitivity of 0.351, a specificity of 0.997, a PPV of 0.812, and an NPV of 0.976 at the same classification threshold.

As shown in FIG. 6A, confusion matrices may be generated at the classification threshold of 0.75 to summarize model predictions compared to expert review labels. At the first institution, the confusion matrix may show 942 true negatives, 32 false negatives, 5 false positives, and 21 true positives, yielding a contamination rate of 5.3%. At the second institution, the confusion matrix may show 960 true negatives, 24 false negatives, 3 false positives, and 13 true positives, yielding a contamination rate of 3.7%. The confusion matrices demonstrate that the machine learning model achieves high specificity while maintaining the ability to identify contaminated specimens in real-world validation sets.

### Performance in Transfusion Setting

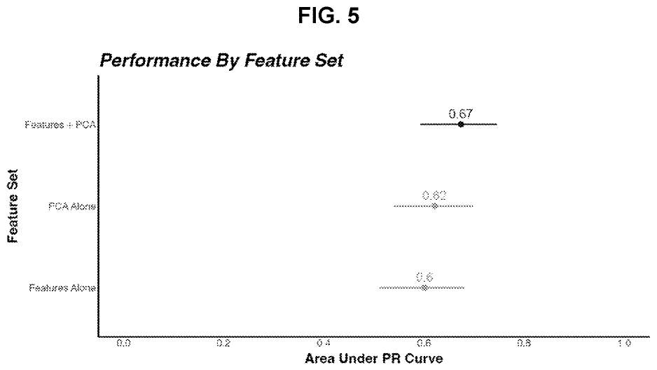

FIG. 7A and FIG. 7B depict receiver operating characteristic (ROC) curves and precision-recall (PR) curves for evaluating machine learning model performance in identifying intravenous fluid contamination in CBC trios where a red blood cell (RBC) transfusion was administered between the current and post samples. Given that the post hemoglobin concentration may be affected by transfusion-induced hemoglobin increases, performance evaluation in the transfusion setting may provide a more comprehensive assessment of model robustness to this common edge case.

In some cases, validation sets may comprise 1000 CBC trios at each institution for which a RBC transfusion was administered between the current and post CBC results. Expert review may provide ground truth labels for contamination status in the transfusion validation sets. In the setting of RBC transfusion, the machine learning model may achieve an area under the receiver operating characteristic curve (auROC) of 0.929 at a first institution and 0.930 at a second institution. The machine learning model may achieve an area under the precision-recall curve (auPR) of 0.688 at the first institution and 0.697 at the second institution.

The ROC curves and PR curves demonstrate the trade-off in labeling performance across classification thresholds for CBC trios where a transfusion was administered between the current and post samples. The area under the curves summarizes the discriminatory potential of the machine learning model in distinguishing contaminated samples from non-contaminated samples in the transfusion setting.

Expert review of transfused patients may reveal contamination rates exceeding 11% in CBC trios associated with transfusion events. In some cases, the contamination rate may be approximately 11.8% at a first institution and approximately 12.5% at a second institution in the transfusion validation sets. The elevated contamination rates observed in transfused patients compared to randomly selected CBC trios may indicate that contamination-induced artificially low hemoglobin measurements contribute to transfusion decisions in a portion of cases.

At a classification threshold of 0.75, the machine learning model may achieve a sensitivity of 0.712, a specificity of 0.922, a positive predictive value (PPV) of 0.549, and a negative predictive value (NPV) of 0.96 at the first institution in the transfusion setting. At the second institution, the machine learning model may achieve a sensitivity of 0.648, a specificity of 0.919, a PPV of 0.533, and an NPV of 0.948 at the same classification threshold. The performance metrics demonstrate that the machine learning model maintains the ability to identify contaminated specimens in CBC trios where a transfusion was administered, despite the confounding effect of transfusion-induced hemoglobin increases on the post sample values.

### Cumulative Impact Analysis by Collector

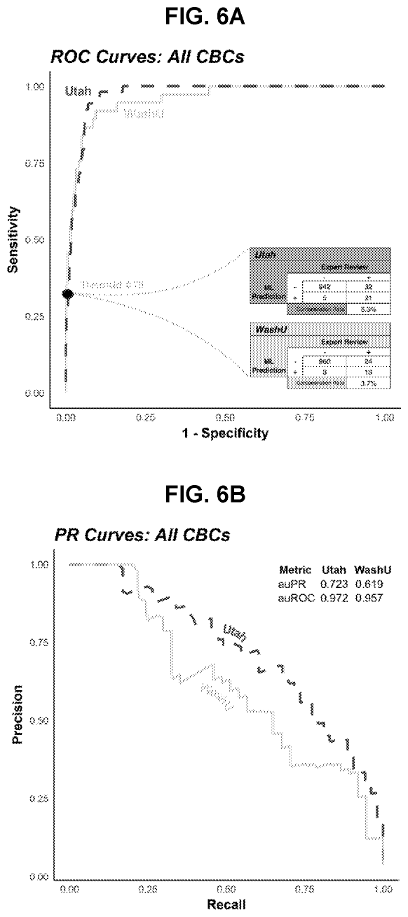

FIG. 8 depicts a graph showing the cumulative impact of line draws by collector. The systems and methods described herein may generate cumulative impact analysis of line draws by collector to identify individuals with higher contamination rates per line draw for targeted quality improvement initiatives.

In some cases, the cumulative impact analysis may track the number of line draws performed by each specimen collector and correlate the line draws with contamination predictions generated by the machine learning model. The cumulative impact may be calculated by aggregating contamination events attributed to each collector over a period of time. As the number of line draws increases for a given collector, the cumulative impact may increase at a rate proportional to the contamination rate associated with that collector.

As shown in FIG. 8, the graph may display multiple curved lines, where each line represents data from a different collector. The lines may show an upward trend as the number of line draws increases, indicating that collectors with more line draws contribute to a greater cumulative impact. In some cases, certain collector lines may rise more steeply than others, suggesting variation in contamination rates among different collectors.

The cumulative impact analysis may enable identification of high-impact individuals whose collection practices result in elevated contamination rates. Collectors with steeper cumulative impact curves may have higher rates of contamination-related events per line draw compared to collectors with shallower curves. In some cases, the cumulative impact analysis may be used to prioritize collectors for targeted intervention, such as educational outreach regarding proper phlebotomy technique.

The systems and methods may generate reports identifying specimen collectors associated with elevated cumulative impact values. In some cases, the reports may rank collectors by cumulative impact or by contamination rate per line draw. The reports may be used by laboratory personnel or quality improvement teams to focus resources on collectors whose practices may benefit from additional training or process modifications.

By tracking cumulative impact over time, the systems and methods may also measure the realized impact of quality improvement efforts. In some cases, a reduction in the slope of a collector's cumulative impact curve following an intervention may indicate improvement in collection practices. The cumulative impact analysis may provide a mechanism for ongoing monitoring of specimen collection quality at an individual collector level.

### Contamination Rate by Collection Unit

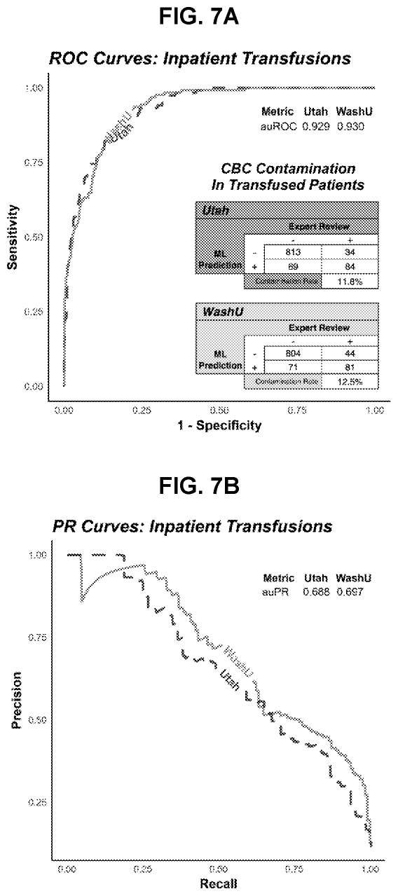

FIG. 9A depicts a graph illustrating contamination rate by collection unit. The systems and methods described herein may generate contamination rate analysis by collection unit to identify departments with elevated contamination rates. The contamination rate analysis may aggregate contamination predictions generated by the machine learning model according to collection unit identifiers associated with each CBC trio.

Collection units within a healthcare facility may include various hospital departments and care areas such as intensive care units, emergency departments, surgical units, and general inpatient wards. Each collection unit may be associated with a contamination rate representing a proportion of CBC trios predicted as contaminated relative to a total number of CBC trios collected within that unit.

As shown in FIG. 9A, each collection unit may be represented by a horizontal bar extending from a vertical axis, with the length of each bar corresponding to the contamination rate for that particular unit. The bars may vary in length across the different collection units, indicating differing contamination rates among the various locations where blood specimens are collected.

The contamination rate analysis by collection unit may enable comparison of intravenous fluid contamination prevalence across different clinical settings within an institution. In some cases, certain collection units may demonstrate higher contamination rates compared to other collection units. The variation in contamination rates across collection units may reflect differences in specimen collection practices, patient populations, or clinical workflows within each unit.

The systems and methods may generate reports identifying collection units associated with elevated contamination rates. In some cases, the reports may rank collection units by contamination rate to facilitate identification of units that may benefit from targeted quality improvement initiatives. Laboratory personnel or quality improvement teams may use the contamination rate analysis to focus resources on collection units whose practices may benefit from additional training or process modifications.

By tracking contamination rates by collection unit, the systems and methods may provide a mechanism for ongoing monitoring of specimen collection quality at a unit level. In some cases, changes in contamination rates over time within a collection unit may indicate the effectiveness of quality improvement interventions implemented within that unit.

### Contamination Severity Estimation

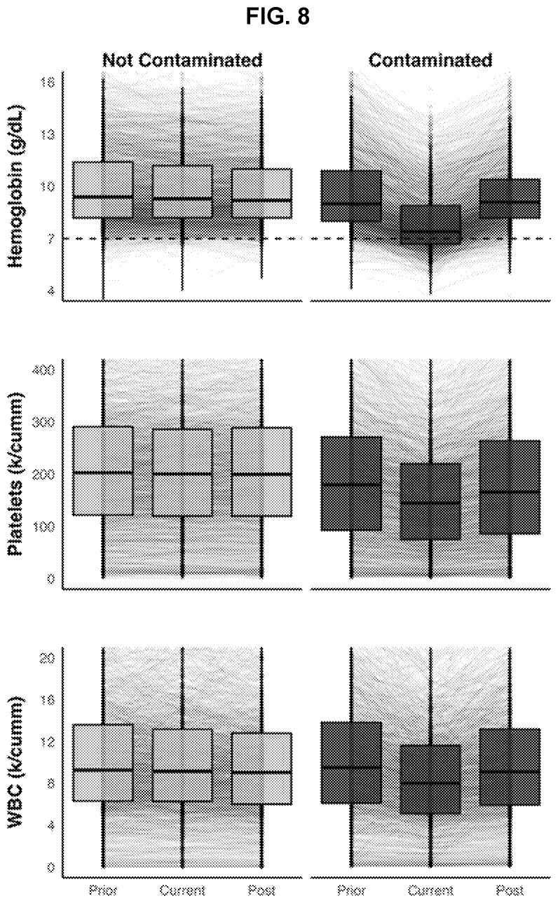

FIG. 10 depicts a graph showing estimated contamination severity in predicted positive complete blood count trios across two institutions. The systems and methods described herein may estimate a contamination severity for each complete blood count trio predicted as contaminated. The contamination severity may comprise an estimated mixture ratio representing a proportion of contaminating fluid in the current sample.

Contamination severity may be estimated using regression models trained on simulated contamination data. As described above, the machine learning model may be trained using simulated contamination data generated by applying linear dilution to analyte values in a training set of complete blood count trios. Regression models trained on the same simulated contamination data may be used to estimate the mixture ratio for each complete blood count trio predicted as contaminated by the classification models.

The mixture ratio represents a proportion of contaminating fluid relative to a physiologic specimen. A mixture ratio of 0 indicates no contamination, while higher mixture ratio values indicate greater proportions of contaminating fluid relative to the physiologic patient specimen. For example, a mixture ratio of 0.20 indicates that approximately 20% of the specimen volume comprises contaminating intravenous fluid.

As shown in FIG. 10, the estimated contamination severity may be visualized for contaminated real-world complete blood count trios. Each data point in FIG. 10 represents one predicted contaminated specimen. The data points may be displayed as two overlapping distributions, with one distribution representing contaminated specimens from a first institution and another distribution representing contaminated specimens from a second institution.

At a first institution, the median mixture ratio for contaminated specimens may be 0.13. At a second institution, the median mixture ratio for contaminated specimens may be 0.14. The distributions of estimated mixture ratios may demonstrate a similar pattern at both institutions, with the majority of contaminated specimens clustered at lower mixture ratios and fewer specimens extending toward higher mixture ratios.

In some cases, the maximum estimated mixture ratio may be 0.60 at the first institution and 0.66 at the second institution. The range of estimated mixture ratios may extend from approximately 0.05 to the maximum values, with the majority of contaminated specimens concentrated between mixture ratios of approximately 0.05 and 0.30.

The distribution of contamination severity across predicted positive complete blood count trios may indicate that contamination events with lower mixture ratios occur more frequently than contamination events with higher mixture ratios. In some cases, a portion of contaminated specimens may have mixture ratios above 0.10, and a smaller portion may have mixture ratios above 0.20.

By estimating contamination severity for each complete blood count trio predicted as contaminated, the systems and methods may provide additional information regarding the magnitude of the dilutional effect in contaminated specimens. The estimated mixture ratio may be used to assess the degree to which measured analyte values in the current sample deviate from the patient's physiologic baseline due to intravenous fluid contamination.

### Orthogonal Validation Using Basic Metabolic Panels

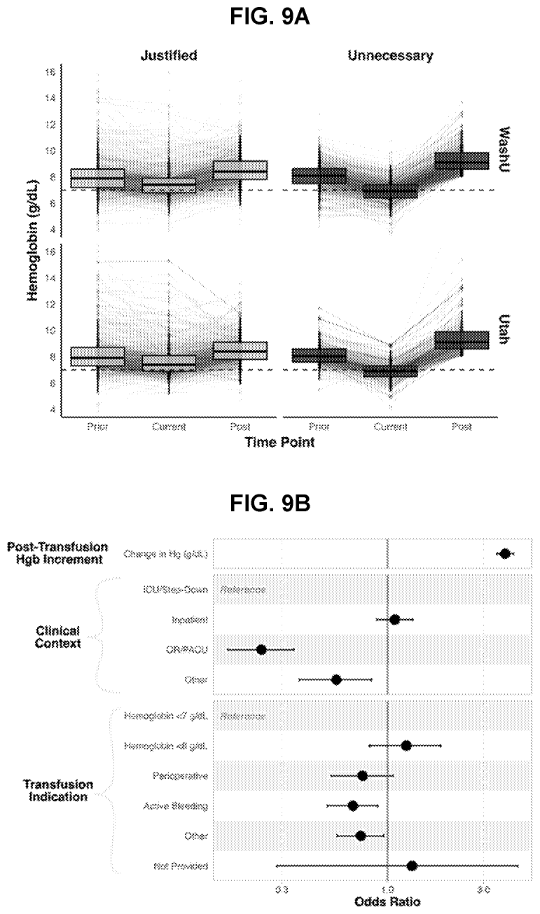

FIG. 11 depicts a scatter plot showing contamination severity in paired basic metabolic panels (BMPs) from contaminated complete blood counts (CBCs). The systems and methods described herein may match a complete blood count trio predicted as contaminated with a corresponding basic metabolic panel collected within a predetermined time window of the current sample to provide orthogonal validation of the contamination prediction.

In some cases, CBC trios predicted as contaminated may be matched to any corresponding BMP that was collected within 5 minutes of the current CBC. The 5-minute time window may be selected to identify BMPs that were collected concurrently with the CBC and are therefore likely to have been exposed to the same contamination event. Given that BMPs may be drawn prior to CBCs during specimen collection, contamination events may be more pronounced in BMPs than in paired CBCs when both specimens are affected by the same contaminating intravenous fluid.

The systems and methods may estimate a mixture ratio for the corresponding basic metabolic panel to provide orthogonal validation of the contamination prediction. Mixture ratios may be estimated in the corresponding BMPs using BMP-specific models trained to detect contamination in chemistry results. The estimated mixture ratios from paired CBCs and BMPs may be compared to evaluate whether contamination predictions in CBCs are supported by evidence of contamination in concurrently collected BMPs.

As shown in FIG. 11, the estimated mixture ratios from paired CBCs and BMPs may be displayed in a scatter plot. The horizontal axis represents the CBC mixture ratio, while the vertical axis represents the BMP mixture ratio. Each point in the scatter plot represents a paired CBC and BMP sample predicted to be contaminated, with darker points corresponding to higher point density.

Of contaminated CBC-BMP pairs with a mixture ratio above 0.10, 82% of BMPs may also demonstrate evidence of contamination. In these contaminated pairs, a positive correlation may be observed between the CBC mixture ratio and the BMP mixture ratio. The coefficient of determination (R²) may be 0.56, quantifying the correlation between the mixture ratios for the CBCs and BMPs. The line of best fit may show a slope of 1.08, indicating that contamination severity in BMPs may be slightly higher on average than in corresponding CBCs.

The correlation between contamination severity in paired CBC and BMP samples provides orthogonal evidence supporting the contamination predictions generated by the machine learning model. The observation that a majority of BMPs collected concurrently with contaminated CBCs also demonstrate evidence of contamination supports the validity of the contamination predictions. The slope of 1.08 in the line of best fit may reflect that BMPs are drawn prior to CBCs during specimen collection, resulting in slightly higher contamination severity in BMPs compared to paired CBCs when both specimens are affected by the same contaminating fluid.

### Feature Importance Analysis

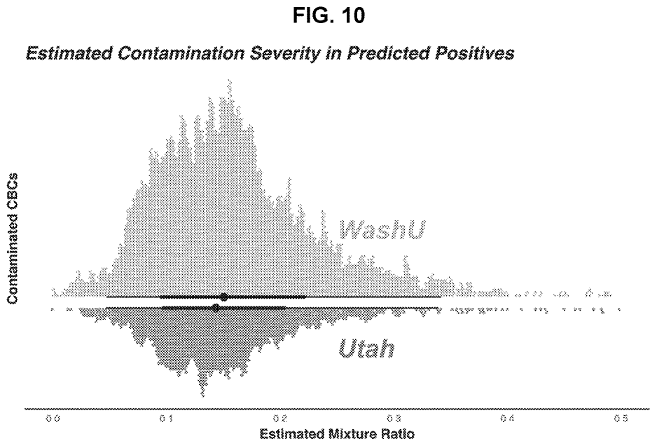

FIG. 12A depicts a feature importance summary plot for a machine learning model used to identify intravenous fluid contamination in complete blood count results. The systems and methods described herein may generate feature importance values using Shapley additive explanation values to identify contributions of individual features to the contamination prediction.

Shapley additive explanation (SHAP) values estimate each individual feature's contribution to the overall predicted probability in relation to all other feature values for each input sample. SHAP values may be aggregated across all predictions to provide a global view of which features exert the strongest influence on model predictions across a full test set. The global evaluation of feature importance using SHAP values may identify features in decreasing order of median magnitude of their contribution to the contamination prediction.

As shown in FIG. 12A, features may be listed in decreasing order of their median contribution magnitude to model predictions. Each point in the summary plot represents one complete blood count trio from the test set. Points to the right of a center line indicate that the feature value increased the model's predicted probability of contamination, while points to the left indicate that the feature value decreased the predicted probability. A color gradient may indicate the feature value for each data point, with lighter points representing trios where that feature had a higher value and darker points representing lower feature values.

In some cases, the global evaluation of feature importance may identify Principal Component 1 (PC1) as having the highest impact on predictions. PC1 represents the transformed projection of the axis of greatest variation in the simulated training data set. Following PC1, the change in hemoglobin from current to post (hemoglobin Δ_{Post}) may have the second highest contribution, followed by the change in hemoglobin from current to prior (hemoglobin Δ_{Prior}).

The SHAP values may reveal that larger decreases from prior measurements and larger increases to post measurements correlate with higher predicted probabilities of contamination. In some cases, lower PC1 values may correlate with higher contamination probabilities. The principal components may effectively capture the anomaly-with-resolution pattern typical of contamination, while higher PC1 values may correspond to an opposite pattern across the analytes.

FIG. 12B depicts the anomaly-with-resolution pattern as a function of principal component tertiles. FIG. 12B may show PC1 and PC2 values for each complete blood count trio, with data points colored by change from prior and change to post for hemoglobin, platelet count, and white blood cell count. In some cases, data points representing increases may be displayed in a first color, while data points representing decreases may be displayed in a second color.

The principal components may capture the anomaly-with-resolution pattern in a dimensionally reduced space. Given that the error-generating process of dilution by contaminating fluid is linear and proportional across all analytes, principal component analysis may effectively capture the resulting anomaly-with-resolution pattern from diluted specimens. PC1 and PC2 may represent two axes that describe the anomaly-with-resolution pattern, with lower PC1 values corresponding to the characteristic dilutional decrease followed by resolution that is indicative of intravenous fluid contamination.

Although the inclusion of principal component analysis may make the machine learning pipeline more complex to explain than if principal component analysis were excluded, the extent to which PC1 effectively captures the anomaly-with-resolution pattern may provide adequate explainability for the resulting pipeline. The congruence between expected and observed feature importances estimated by SHAP values may support confidence in the model's predictions.

### Unnecessary Transfusion Rate Analysis

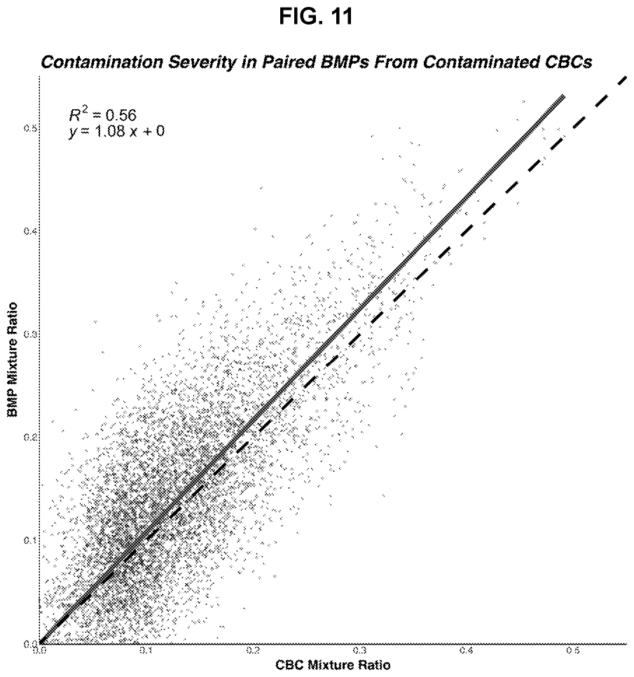

FIG. 13 depicts a graph showing unnecessary transfusion rates by classification threshold. The systems and methods described herein may calculate an unnecessary transfusion rate based on a ratio of transfusion events classified as potentially unnecessary to a total number of transfusion events for which a complete blood count trio is available. The unnecessary transfusion rate may vary depending on the classification threshold applied to the contamination prediction.

As described above, a transfusion event may be classified as potentially unnecessary when the complete blood count trio associated with the transfusion event is predicted as contaminated, a post-transfusion hemoglobin concentration exceeds a threshold hemoglobin value, and the post-transfusion hemoglobin concentration exceeds a prior hemoglobin concentration. In some cases, the threshold hemoglobin value may be 8 g/dL.

The rule set for labeling transfusions as potentially unnecessary may be validated against expert chart review. In some cases, positive predictions generated by the machine learning model may be compared to expert chart review to evaluate the feasibility of applying the criteria for labeling transfusions as potentially unnecessary. At a first institution, 81 of 84 transfusions administered after positive predictions may be deemed potentially unnecessary by manual review. At a second institution, 77 of 81 transfusions administered after positive predictions may be deemed potentially unnecessary by manual review. The rule set for labeling transfusions as potentially unnecessary may achieve a combined positive predictive value of 96% when validated against expert chart review.

Transfusions deemed justified despite predicted contamination may include scenarios in which the post-transfusion hemoglobin concentration was below 8 g/dL after the transfusion, active intraoperative or postoperative bleeding was present, or acute symptoms consistent with anemia were present. The hemoglobin concentration threshold was based on a clinical study analyzing hemoglobin-based transfusion thresholds. See Carson et al., J. Am. Med. Assoc. 330(19): 1892-1902 (2023).

As shown in FIG. 13, the implied unnecessary transfusion rate may vary as a function of the classification threshold. At a first institution, 7,632 inpatient red blood cell transfusions may have a matched complete blood count trio available for contamination prediction. At a second institution, 27,980 inpatient red blood cell transfusions may have a matched complete blood count trio available for contamination prediction. The proportion of transfusions flagged as positive may vary depending on the classification threshold selected.

In some cases, the potentially unnecessary transfusions may represent 6% to 9% of all inpatient transfusions for which a complete blood count trio is available. The denominator of transfusions with a matched complete blood count trio may capture the population at risk for misguided transfusion decisions due to contamination. In some cases, 686 transfusions (9%) at a first institution and 1,690 transfusions (6%) at a second institution may be flagged as potentially unnecessary using the rule set.

The systems and methods may generate reports identifying the implied unnecessary transfusion rate at different classification thresholds. In some cases, the reports may display the relationship between classification threshold and implied unnecessary transfusion rate to support selection of an appropriate threshold for a given clinical context. The analysis of unnecessary transfusion rates by classification threshold may inform quality improvement initiatives aimed at reducing transfusions motivated by contamination-induced artificially low hemoglobin measurements.

### Cumulative Impact of Line Draws by Collector

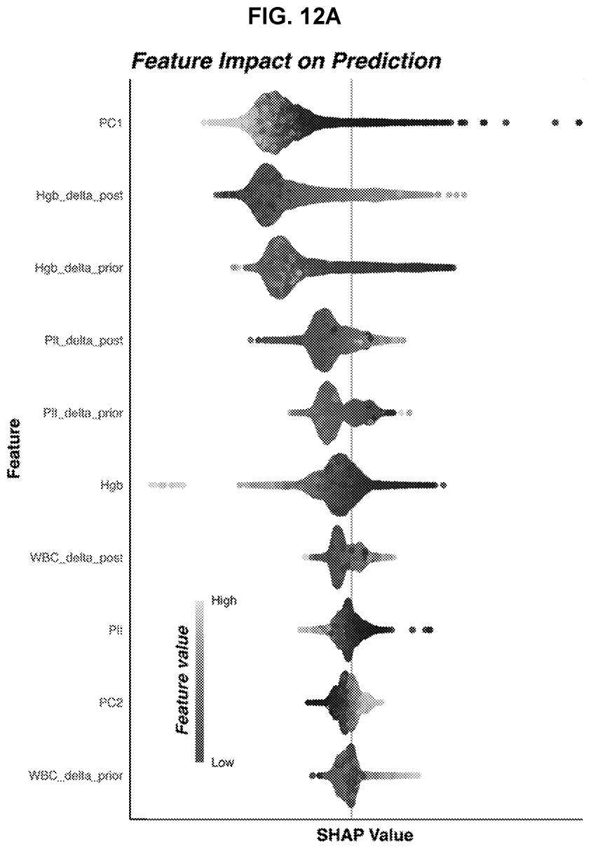

FIG. 14 depicts a graph showing the cumulative impact of line draws by collector. The systems and methods described herein may generate cumulative impact analysis of line draws by collector to identify individuals with higher contamination rates per line draw for targeted quality improvement initiatives. As shown in FIG. 14, the graph may display multiple curved lines, where each line represents data from a different collector. The lines may show an upward trend as the number of line draws increases, indicating that collectors with more line draws contribute to a greater cumulative impact. In some cases, certain collector lines may rise more steeply than others, suggesting variation in contamination rates among different collectors. The cumulative impact analysis may enable identification of high-impact individuals whose collection practices result in elevated contamination rates.

### Contamination Rate by Phlebotomist

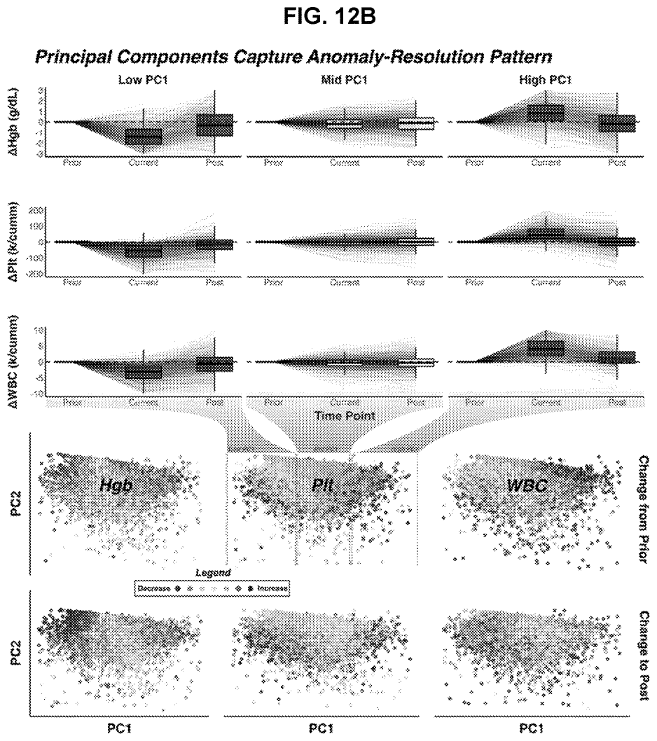

FIG. 15 depicts a graph showing monthly contamination rate by phlebotomist. The systems and methods described herein may aggregate contamination predictions by phlebotomist to generate monthly contamination rates by individual collector for quality improvement analysis.

In some cases, the systems and methods may calculate a contamination rate for each phlebotomist based on a ratio of complete blood count trios predicted as contaminated to a total number of complete blood count trios collected by that phlebotomist during a given time period. The contamination rate may be expressed as a percentage representing the proportion of specimens collected by a phlebotomist that are predicted to be contaminated by intravenous fluid.

As shown in FIG. 15, contamination rates may vary among phlebotomists. Each data point in FIG. 15 may represent a contamination rate for a particular phlebotomist, displayed with confidence intervals indicating the associated uncertainty range. The data points may be shaded according to the number of collections performed by each phlebotomist, with different shading indicating higher or lower collection volumes.

In some cases, phlebotomists with higher collection volumes may have narrower confidence intervals around their contamination rate estimates compared to phlebotomists with lower collection volumes. The distribution of contamination rates across phlebotomists may show that most phlebotomists have contamination rates clustered within a range, with some phlebotomists demonstrating contamination rates that extend toward higher values.

The systems and methods may generate reports identifying phlebotomists associated with elevated contamination rates. In some cases, the reports may rank phlebotomists by contamination rate to facilitate identification of individuals whose collection practices may benefit from targeted quality improvement initiatives. Laboratory personnel or quality improvement teams may use the monthly contamination rate analysis to focus resources on phlebotomists whose practices may benefit from additional training regarding proper phlebotomy technique.

By tracking contamination rates by phlebotomist over time, the systems and methods may provide a mechanism for ongoing monitoring of specimen collection quality at an individual collector level. In some cases, changes in contamination rates over time for a given phlebotomist may indicate the effectiveness of quality improvement interventions directed toward that individual. The monthly aggregation of contamination predictions by phlebotomist may enable identification of trends in collection quality and support data-driven quality improvement initiatives.

### Unnecessary Transfusion Rate by Collection Unit

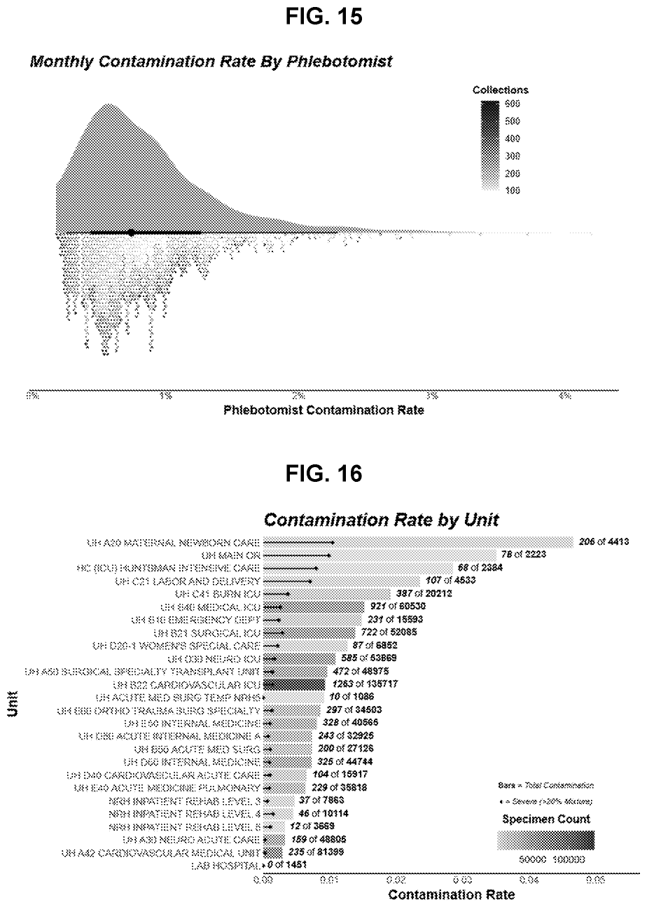

FIG. 17 depicts a bar graph showing estimated unnecessary transfusion rate by collecting unit. The systems and methods described herein may generate an estimated unnecessary transfusion rate by collecting unit for targeted quality improvement initiatives. The estimated unnecessary transfusion rate by collecting unit may aggregate transfusion events classified as potentially unnecessary according to collection unit identifiers associated with each transfusion event.

Collection units within a healthcare facility may include various hospital departments and care areas such as intensive care units (ICUs), step-down units, inpatient wards, operating rooms (ORs), post-anesthesia care units (PACUs), and other clinical settings. Each collection unit may be associated with an estimated unnecessary transfusion rate representing a proportion of transfusion events classified as potentially unnecessary relative to a total number of transfusion events within that unit.

As shown in FIG. 17, each collection unit may be represented by a horizontal bar extending from a vertical axis, with the length of each bar corresponding to the estimated unnecessary transfusion rate for that particular unit. The bars may vary in length across the different collection units, indicating differing proportions of unnecessary transfusions among the various locations where transfusion events occur. In some cases, the bars may be displayed with shading or coloring based on the absolute number of unnecessary transfusions within each unit.

The systems and methods may identify that transfusions administered in OR/PACU settings are less likely to be labeled as potentially unnecessary compared to transfusions administered in ICU or inpatient ward settings. In some cases, transfusions administered peri-operatively or intra-operatively may demonstrate lower unnecessary transfusion rates compared to transfusions administered in other clinical contexts. The variation in unnecessary transfusion rates across clinical contexts may reflect differences in clinical decision-making, patient acuity, or the presence of active clinical indications for transfusion in operative settings.

The systems and methods may identify that transfusions with an active bleeding indication are less likely to be labeled as potentially unnecessary. In some cases, transfusions for which the clinician-provided indication is active bleeding may demonstrate lower rates of classification as potentially unnecessary compared to transfusions with other indications. The presence of active bleeding may represent a clinical scenario in which transfusion is more likely to be clinically indicated regardless of potential contamination in the pre-transfusion complete blood count specimen.

The systems and methods may identify that larger post-transfusion hemoglobin increments are more likely to be labeled as potentially unnecessary. In some cases, transfusion events classified as potentially unnecessary may demonstrate a median change in hemoglobin concentration from current to post of approximately 2.2 to 2.3 g/dL, compared to a median change of approximately 0.9 to 1.0 g/dL for transfusion events classified as justified. The larger post-transfusion hemoglobin increments observed in potentially unnecessary transfusions may reflect that the pre-transfusion hemoglobin measurement was artificially decreased due to intravenous fluid contamination, resulting in a greater apparent increase following transfusion when the post-transfusion specimen is properly collected.

The estimated unnecessary transfusion rate by collecting unit may enable comparison of unnecessary transfusion prevalence across different clinical settings within an institution. In some cases, certain collection units may demonstrate higher estimated unnecessary transfusion rates compared to other collection units. The variation in estimated unnecessary transfusion rates across collection units may inform targeted quality improvement initiatives aimed at reducing transfusions motivated by contamination-induced artificially low hemoglobin measurements.

The systems and methods may generate reports identifying collection units associated with elevated estimated unnecessary transfusion rates. In some cases, the reports may rank collection units by estimated unnecessary transfusion rate to facilitate identification of units that may benefit from targeted quality improvement initiatives. Laboratory personnel, transfusion medicine specialists, or quality improvement teams may use the estimated unnecessary transfusion rate analysis to focus resources on collection units whose practices may benefit from additional training or process modifications.

### Method for Identifying Potentially Unnecessary Transfusions

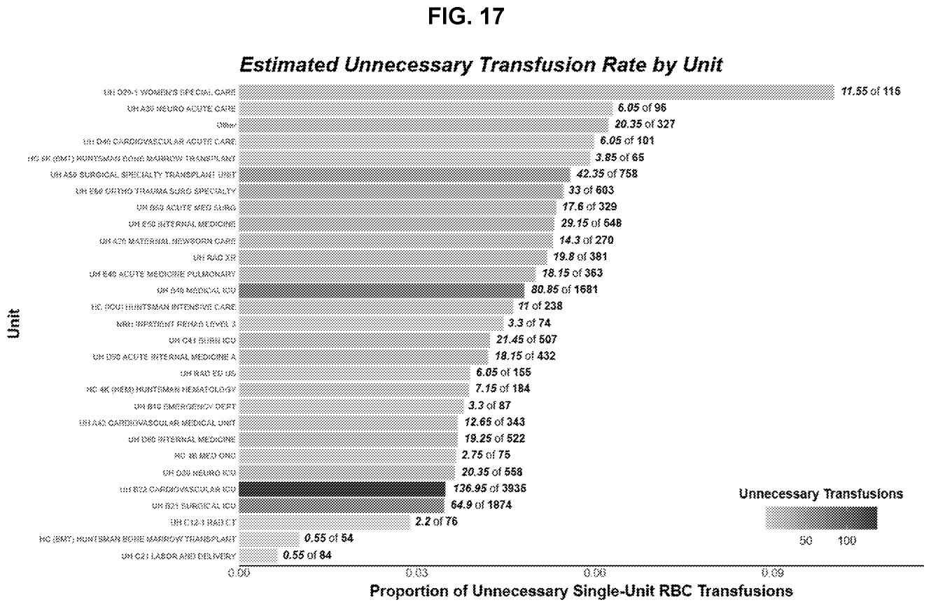

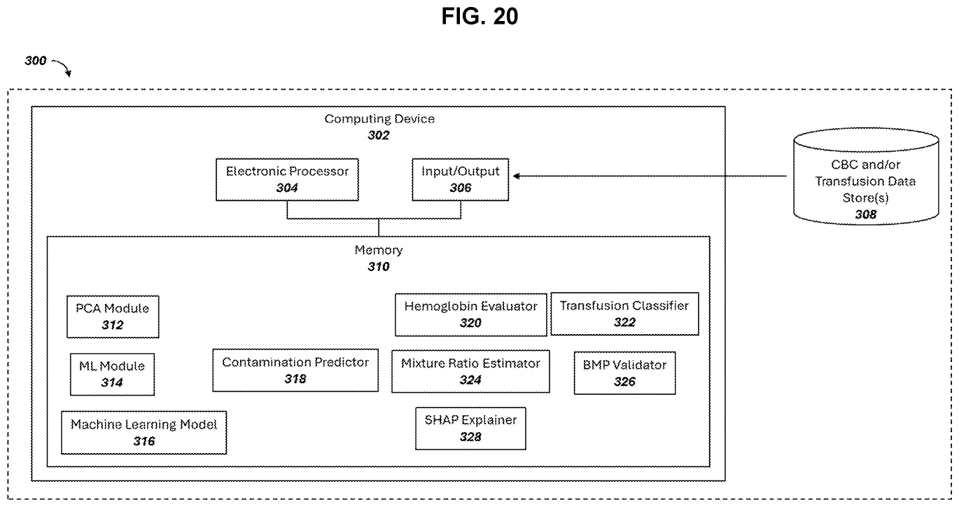

FIG. 18 illustrates a flowchart of a method 100 for identifying potentially unnecessary red blood cell transfusions. Method 100 may be performed by one or more electronic processors executing instructions stored in a memory, including, for example, the electronic processor 304 and memory 310 described with respect to the system 300 in FIG. 20.

Method 100 includes receiving (at block 102), by one or more electronic processors, CBC and/or transfusion data comprising a plurality of red blood cell transfusion events. Each transfusion event may be associated with a complete blood count trio comprising a prior sample collected before a current sample, the current sample collected before the transfusion event, and a post sample collected after the transfusion event. The prior sample, the current sample, and the post sample may each be associated with a hemoglobin concentration value, a platelet count value, and a white blood cell count value. In some cases, the prior sample and the post sample are collected within 48 hours of the current sample.

The CBC and/or transfusion data may be extracted from blood product administration modules of an electronic medical record system. The one or more electronic processors performing the method 100 may be part of or separate from such an electronic medical record system. When separate, the one or more electronic processors are configured to communicate with the electronic medical record system over one or more communication channels or networks (e.g., wired, wireless, or a combination thereof) and such communication may use an application programming interface (API) call or other type of data request or query. In some cases, the CBC and/or transfusion data may comprise single-unit red blood cell transfusions administered to inpatients. Each transfusion event may be matched to a corresponding complete blood count trio based on the timing of specimen collection relative to the transfusion administration.

Method 100 also includes applying (at block 104), by the one or more electronic processors, a machine learning model to each complete blood count trio to generate a contamination prediction indicating a likelihood that the current sample is contaminated by intravenous fluid. As described above, the machine learning model may be trained to identify an anomaly-with-resolution pattern in complete blood count results indicative of intravenous fluid contamination. The contamination prediction may be expressed as a probability value between 0 and 1, where higher values indicate a greater likelihood of contamination. In some cases, a classification threshold may be applied to the contamination prediction to generate a binary classification indicating whether the current sample is contaminated.

Method 100 further includes evaluating (at block 106), by the one or more electronic processors, for each transfusion event associated with a complete blood count trio predicted as contaminated, whether a post-transfusion hemoglobin concentration exceeds a threshold hemoglobin value and whether the post-transfusion hemoglobin concentration exceeds a prior hemoglobin concentration. The post-transfusion hemoglobin concentration may correspond to the hemoglobin concentration value from the post sample in the complete blood count trio. The prior hemoglobin concentration may correspond to the hemoglobin concentration value from the prior sample in the complete blood count trio.

In some cases, the threshold hemoglobin value is 8 g/dL. The threshold hemoglobin value of 8 g/dL may be selected to remove cases for which empiric transfusion may be justified by clinical practice guidelines. See Carson et al., J. Am. Med. Assoc. 330(19): 1892-1902 (2023). By evaluating whether the post-transfusion hemoglobin concentration exceeds the prior hemoglobin concentration, the method 100 (at block 106) may remove cases for which hemoglobin decrease could have occurred for clinically significant reasons, such as acute blood loss.

Method 100 includes classifying (at block 108), by the one or more electronic processors, a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration. A transfusion event may be classified as potentially unnecessary when all three conditions are fulfilled. This classification may be stored in memory and may be included in one or more reports or notifications, including electronic reports (e.g., output as part of a graphical user interface), electronic notifications (e.g., email messages, text messages, application notifications, chat messages, or the like), or a combination thereof. The classification may be aggregated and/or analyzed as described below.

The classification of a transfusion event as potentially unnecessary may indicate that the transfusion was motivated by dilutional effects of intravenous fluid contamination rather than by clinically indicated factors. In some cases, the artificially low hemoglobin measurement resulting from intravenous fluid contamination in the current sample may have prompted a transfusion that would not otherwise be clinically indicated. When the post-transfusion hemoglobin concentration exceeds both the threshold hemoglobin value and the prior hemoglobin concentration, the transfusion event may be identified as one that was administered based on an erroneously low pre-transfusion hemoglobin measurement.

In some cases, transfusion events classified as potentially unnecessary may demonstrate larger post-transfusion hemoglobin increments compared to transfusion events classified as justified. The larger post-transfusion hemoglobin increments observed in potentially unnecessary transfusions may reflect that the pre-transfusion hemoglobin measurement was artificially decreased due to intravenous fluid contamination, resulting in a greater apparent increase following transfusion when the post-transfusion specimen is properly collected.

The rule set for classifying transfusions as potentially unnecessary may be validated against expert chart review. In some cases, transfusions deemed justified despite predicted contamination may include scenarios in which the post-transfusion hemoglobin concentration was below 8 g/dL after the transfusion, active intraoperative or postoperative bleeding was present, or acute symptoms consistent with anemia were present.

### Method for Detecting IV Fluid Contamination

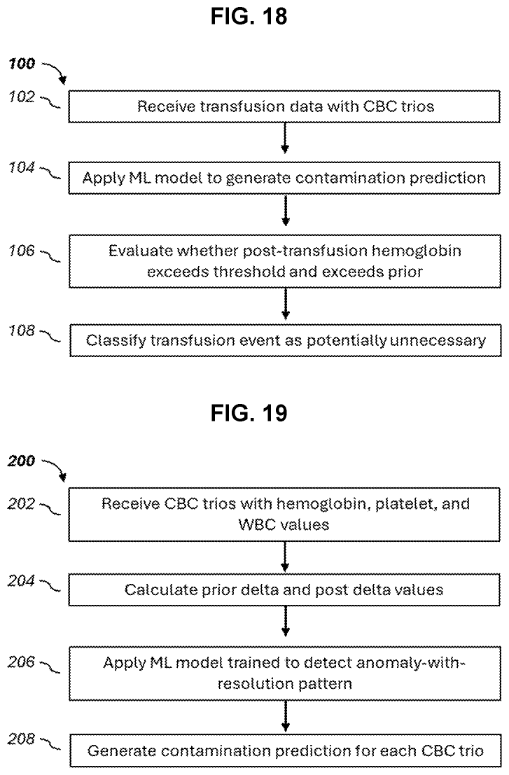

FIG. 19 illustrates a flowchart of a method 200 for detecting intravenous fluid contamination in complete blood count results. Method 200 may be performed by one or more electronic processors executing instructions stored in a memory, including, for example, the electronic processor 304 and memory 310 described with respect to the system 300 in FIG. 20.

Method 200 includes receiving (at block 202), by one or more electronic processors, a plurality of complete blood count trios (e.g., from one or more enterprise data warehouses, laboratory information systems, or a combination thereof as described below). Each complete blood count trio comprises a prior sample, a current sample, and a post sample. The prior sample may be associated with a first hemoglobin concentration value, a first platelet count value, and a first white blood cell count value. The current sample may be associated with a second hemoglobin concentration value, a second platelet count value, and a second white blood cell count value. The post sample may be associated with a third hemoglobin concentration value, a third platelet count value, and a third white blood cell count value.

In some cases, the prior sample and the post sample are collected within 48 hours of the current sample. The 48-hour time window may be selected to capture sequential laboratory measurements that are temporally proximate to the current sample while allowing sufficient time for specimen collection in clinical workflows. Complete blood count trios that lack prior or post measurements within the predetermined time window may be excluded from analysis.

Method 200 also includes calculating (at block 204), by the one or more electronic processors, for each complete blood count trio, delta values representing changes in analyte values between samples. This calculation (at block 204) may include calculating a prior delta comprising a difference between the first hemoglobin concentration value and the second hemoglobin concentration value, a difference between the first platelet count value and the second platelet count value, and a difference between the first white blood cell count value and the second white blood cell count value. This calculation (at block 204) may further include calculating a post delta (Δ_{Post}) comprising a difference between the third hemoglobin concentration value and the second hemoglobin concentration value, a difference between the third platelet count value and the second platelet count value, and a difference between the third white blood cell count value and the second white blood cell count value. In some examples, the difference values may be received as input such that the electronic processors, as part of method 200, do not need to perform the calculations (or a portion thereof) at this part of the method 200 and may, in some situations, receive the delta values and the values for the current sample.

The prior delta values (Δ_{Prior}) capture changes in analyte concentrations from the prior sample to the current sample, while the post delta values capture changes from the current sample to the post sample. In the anomaly-with-resolution pattern indicative of intravenous fluid contamination, the prior delta values may reflect a decrease in analyte concentrations from the prior sample to the current sample due to dilution, while the post delta values may reflect an increase in analyte concentrations from the current sample to the post sample as the measured values rebound toward the patient's baseline when a subsequent specimen is properly collected.

Method 200 further includes applying (at block 206), by the one or more electronic processors, a machine learning model to the plurality of complete blood count trios (or data calculated therefrom). The machine learning model may be trained to distinguish contaminated samples from non-contaminated samples based on an anomaly-with-resolution pattern. As described above, the anomaly-with-resolution pattern comprises a characteristic pattern in which count and concentration results in a contaminated specimen initially decrease due to dilution, then rebound toward the patient's baseline when a subsequent specimen is properly collected.

In some cases, the machine learning model comprises a gradient boosting model. The gradient boosting model may be implemented using LightGBM or similar frameworks. The gradient boosting model may be tuned using cross-validation to optimize performance metrics such as log-loss. In some cases, the gradient boosting model may use short tree depths and high splitting fractions to add robustness against overfitting from simulated training data.

The machine learning model may receive input features comprising the second hemoglobin concentration value, the second platelet count value, the second white blood cell count value, the prior delta, and the post delta. In some cases, principal component analysis may be performed on the input features to generate one or more principal components, and both raw feature values and the one or more principal components may be provided as inputs to the machine learning model.

Method 200 also includes generating (at block 208), by the one or more electronic processors, for each complete blood count trio, a contamination prediction indicating a likelihood that the current sample is contaminated by intravenous fluid. The contamination prediction may be expressed as a probability value between 0 and 1, where higher values indicate a greater likelihood of contamination.

In some cases, a classification threshold may be applied to the contamination prediction to generate a binary classification indicating whether the current sample is contaminated. The classification threshold may be selected to achieve a positive predictive value exceeding a predetermined value. In some cases, a classification threshold of 0.75 may be applied to achieve a positive predictive value exceeding 0.80.

The contamination predictions generated by method 200 may be aggregated by specimen collector identifier or collection unit identifier to generate contamination rate data for quality improvement analysis. In some cases, the contamination predictions may be used to identify transfusion events associated with complete blood count trios predicted as contaminated, as described above with respect to method 100. The contamination predictions may be stored in memory and may be included in one or more reports or notifications, including electronic reports (e.g., output as part of a graphical user interface), electronic notifications (e.g., email messages, text messages, application notifications, chat messages, or the like), or a combination thereof. The contamination predictions may be aggregated and/or analyzed as described herein. The prediction of contamination could be used to trigger scheduling for additional blood tests (e.g., a CBC) or scheduling a clinical follow-up evaluation.

### System Architecture

FIG. 20 illustrates a system diagram of a system 300 for identifying potentially unnecessary red blood cell transfusions (e.g., method 100), detecting intravenous fluid contamination in complete blood count results (e.g., method 200), or a combination thereof. System 300 includes a computing device 302 and a CBC and/or transfusion data store(s) 308. In some examples, the computing system 302 includes an electronic processor 304 (e.g., a microprocessor, application specific integrated circuit, etc.), and a memory 310. The memory 310 may include non-transitory computer-readable media. For example, the memory 310 may include one type or combinations of different types of memory, such as read-only memory (“ROM”), random access memory (“RAM”), electrically erasable programmable read-only memory (“EEPROM”), flash memory, or other suitable memory devices. The electronic processor 304 is coupled to the memory 310 and the input/output interface 306 (e.g., over a bus, wired connection, wireless connection, or a combination thereof). The electronic processor 304 sends and receives information (for example, from the memory 310 and/or the input/output interface 306) and processes the information by executing one or more software instructions or modules stored in the memory 310 or another non-transitory computer readable medium. The software can include firmware, one or more applications, program data, filters, rules, one or more program modules, and other executable instructions. In some examples, the electronic processor 304 is configured to retrieve from the memory 310 and execute, among other things, software for performing the functionality described herein. For example, the memory 310 may store data instructions that, when executed by electronic processor 304, cause the system 300 to perform operations for detecting intravenous fluid contamination in complete blood count results, classifying transfusion events as potentially unnecessary, or a combination thereof.

The computing device 302 also includes an input/output interface 306 for communicating with external devices, systems, and communication channels (e.g., wired or wireless connections or network, such as, for example, a local area network, a wide area network, or a combination thereof). As illustrated in FIG. 20, the input/output interface 306 may communicate with the CBC and/or transfusion data store(s) 308 for receiving CBC and/or transfusion data comprising a plurality of red blood cell transfusion events. As described herein, each transfusion event may be associated with a complete blood count trio comprising a prior sample collected before a current sample, the current sample collected before the transfusion event, and a post sample collected after the transfusion event. The CBC and/or transfusion data store(s) 308 may provide CBD data, comprising complete blood count trios and/or transfusion data, which may be stored to the memory 310 for processing (e.g., via the method 100). In some cases, the CBC and transfusion data store 308 may receive CBD and/or transfusion data extracted from blood product administration modules of an electronic medical record system.

Alternatively or in addition, the input/output interface 306 may communicate with the transfusion data store or other data stores or data streams to obtaining complete blood count (CRC) trios. Such trios may similarly be stored to the memory 310 for processing (e.g., via the method 200.

It should be understood that the system 300 may include more, fewer, or different components than the components illustrated in FIG. 20 and the components of the system 300 may be combined and distributed in various configurations. For example, in some embodiments, the computing device 302 includes multiple electronic processors, multiple memory modules, multiple input/output interfaces, or a combination thereof. The computing device 302 may also include one or more human-machine interfaces, such as, for example, a keyboard, cursor-controlled device, keypad, microphone, display, touchscreen, speaker, or a combination thereof or receiving input from a user, providing output to a user, or a combination thereof.

As noted, the memory 310 may store instructions that, when executed by electronic processor 304, cause system 300 to perform the method 100, the method 200, or a combination thereof. For example, as illustrated in FIG. 20, the memory 310 may store a PCA module 312, a ML module 314, a ML model 316, a contamination predictor (CP) module 318, a hemoglobin evaluator (HE) module 320, a transfusion classifier (TC) module 322, a SHAP explainer module 328, a mixture ratio estimator (MRE) module 324, and a BMP validator module 326. It should be understood that the software modules illustrated in FIG. 20 may be combined and distributed in various ways and the system 300 may be configured to perform additional functionality than the functionality described herein. Also, in some examples, the memory 310 may only store a subset of the modules illustrated in FIG. 20 (e.g., depending on whether the system 300 is configured to perform the method 100, the method 200, or both.

The PCA module 312 performs principal component analysis on input features to generate one or more principal components. The input features may comprise a hemoglobin concentration value, a platelet count value, and a white blood cell count value from the current sample, a prior delta comprising differences between analyte values of the prior sample and the current sample, and a post delta comprising differences between analyte values of the post sample and the current sample. The PCA module 312 may generate one or more principal components that capture the anomaly-with-resolution pattern indicative of intravenous fluid contamination. Given that the error-generating process of dilution by contaminating fluid is linear and proportional across all analytes, principal component analysis may effectively capture the resulting anomaly-with-resolution pattern from diluted specimens.

The instructions stored in memory 310 (e.g., part of the ML module 314) may cause system 300 to provide both raw feature values and the one or more principal components as inputs to the machine learning model 316. In some cases, the combined approach using both raw feature values and principal components may demonstrate improved performance compared to using either features alone or principal components alone. Residual biological and analytical variation may remain after the variance in the dilutional effect is captured by principal components, and including both raw feature values and principal components may capture both the dilutional pattern and residual variation.

As noted, the machine learning model (ML model) 316 is trained to generate contamination predictions for complete blood count trios. The ML model 316 may comprise a gradient boosting model tuned using cross-validation. In some cases, ML model 316 comprises a LightGBM (Light Gradient Boosting Machine) model, which is a gradient boosting framework. The ML model 316 may be tuned to log-loss in cross-validation, such as 5-fold cross-validation, to optimize performance metrics. In some cases, short tree depths and high splitting fractions may be used in the ML model 316 to add robustness against overfitting from simulated training data.

As described above, the ML model 316 may be trained using simulated contamination data generated by applying linear dilution to analyte values in a training set of complete blood count trios. The linear dilution simulation may be validated using serial dilutions of real-world patient specimens. Contamination severities may be drawn from a distribution of mixture ratios that mimics real-world contamination events. In some cases, contamination may be simulated in 50% of complete blood count trios in the training set, with all non-simulated trios labeled negative for contamination.

The ML model 316 receives input features generated via the PCA module 312, including both raw feature values and one or more principal components. The raw feature values may comprise a hemoglobin concentration value, a platelet count value, and a white blood cell count value from the current sample, a prior delta comprising differences between analyte values of the prior sample and the current sample, and a post delta comprising differences between analyte values of the post sample and the current sample. The ML model 316 may be trained to detect an anomaly-with-resolution pattern indicative of intravenous fluid contamination in complete blood count results.

The contamination predictor module 318 generates contamination predictions based on outputs from the ML model 316. The contamination predictor module 318 generates contamination predictions indicating a likelihood that the current sample in a complete blood count trio is contaminated by intravenous fluid. The contamination prediction may be expressed as a probability value between 0 and 1, where higher values indicate a greater likelihood of contamination. In some cases, the contamination predictor module 318 may apply a classification threshold to the contamination prediction to generate a binary classification indicating whether the current sample is contaminated. A classification threshold of 0.75 in the model output may correspond to achieving a positive predictive value of greater than 0.80.

As part of the method 100, the contamination predictor module 318 provides contamination predictions to the hemoglobin evaluator module 320 for evaluation of transfusion events associated with complete blood count trios predicted as contaminated. As part of the method 100 or 200, the contamination predictor module 318 may also provide contamination predictions to the mixture ratio estimator module 324 for estimation of contamination severity and to the BMP validator 326 for orthogonal validation using paired basic metabolic panels.

The shapley additive explanation (SHAP) explainer module 328 generates feature importance values using Shapley additive explanation values to identify contributions of individual features to the contamination prediction. The SHAP explainer module 328 estimates each individual feature's contribution to the overall predicted probability in relation to all other feature values for each input sample. SHAP values generated by the SHAP explainer module 328 may be aggregated across all predictions to provide a global view of which features exert the strongest influence on model predictions across a full test set.

In some cases, the SHAP explainer module 328 may identify Principal Component 1 (PC1) as having the highest impact on predictions. PC1 represents the transformed projection of the axis of greatest variation in the simulated training data set. Following PC1, the change in hemoglobin from current to post (hemoglobin delta post) may have the second highest contribution, followed by the change in hemoglobin from current to prior (hemoglobin delta prior). The SHAP explainer module 328 may reveal that larger decreases from prior measurements and larger increases to post measurements correlate with higher predicted probabilities of contamination. In some cases, lower PC1 values may correlate with higher contamination probabilities, as the principal components may effectively capture the anomaly-with-resolution pattern typical of contamination.

The SHAP explainer module 328 may provide both local explanations for individual predictions and global explanations aggregated across multiple predictions. The global evaluation of feature importance using SHAP values may identify features in decreasing order of median magnitude of their contribution to the contamination prediction. The congruence between expected and observed feature importances estimated by the SHAP explainer module 328 may support confidence in the predictions generated by the ML model 316.

As part of the method 100, the hemoglobin evaluator module 320 evaluates whether a post-transfusion hemoglobin concentration exceeds a threshold hemoglobin value and whether the post-transfusion hemoglobin concentration exceeds a prior hemoglobin concentration. Hemoglobin evaluator module 320 receives contamination predictions from contamination predictor module 318 and identifies transfusion events associated with complete blood count trios predicted as contaminated. For each transfusion event associated with a complete blood count trio predicted as contaminated, the hemoglobin evaluator module 320 evaluates the hemoglobin concentration values from the prior sample and the post sample in the complete blood count trio.

The post-transfusion hemoglobin concentration may correspond to the hemoglobin concentration value from the post sample in the complete blood count trio. The prior hemoglobin concentration may correspond to the hemoglobin concentration value from the prior sample in the complete blood count trio. In some cases, the threshold hemoglobin value may be 8 g/dL. The threshold hemoglobin value of 8 g/dL may be selected to remove cases for which empiric transfusion may be justified by clinical practice guidelines.

The hemoglobin evaluator module 320 may evaluate whether the post-transfusion hemoglobin concentration exceeds the threshold hemoglobin value. The hemoglobin evaluator module 320 may further evaluate whether the post-transfusion hemoglobin concentration exceeds the prior hemoglobin concentration. By evaluating whether the post-transfusion hemoglobin concentration exceeds the prior hemoglobin concentration, the hemoglobin evaluator module 320 may remove cases for which hemoglobin decrease could have occurred for clinically significant reasons, such as acute blood loss.

As part of the method 100, the transfusion classifier module 322 classifies transfusion events as potentially unnecessary based on the evaluation performed by the hemoglobin evaluator module 320. The transfusion classifier module 322 receives evaluation results from the hemoglobin evaluator module 320 and classifies a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration.

The transfusion classifier module 322 may classify a transfusion event as potentially unnecessary when all three conditions are fulfilled. The classification of a transfusion event as potentially unnecessary may indicate that the transfusion was motivated by dilutional effects of intravenous fluid contamination rather than by clinically indicated factors. In some cases, the artificially low hemoglobin measurement resulting from intravenous fluid contamination in the current sample may have prompted a transfusion that would not otherwise be clinically indicated.

In some cases, transfusion events classified as potentially unnecessary by the transfusion classifier module 322 may demonstrate larger post-transfusion hemoglobin increments compared to transfusion events classified as justified. The larger post-transfusion hemoglobin increments observed in potentially unnecessary transfusions may reflect that the pre-transfusion hemoglobin measurement was artificially decreased due to intravenous fluid contamination, resulting in a greater apparent increase following transfusion when the post-transfusion specimen is properly collected.

The transfusion classifier module 322 may generate classification outputs indicating whether each transfusion event is classified as potentially unnecessary or justified. In some cases, the transfusion classifier module 322 may provide classification outputs to downstream components for aggregation and reporting. The classification outputs generated by the transfusion classifier module 322 may be aggregated by specimen collector identifier, collection unit identifier, or clinical context to support quality improvement analysis.

The mixture ratio estimator module 324 estimates a contamination severity for each complete blood count trio predicted as contaminated. The contamination severity comprises an estimated mixture ratio representing a proportion of contaminating fluid relative to a physiologic patient specimen. The mixture ratio may be expressed as a value between 0 and 1, where a mixture ratio of 0 indicates no contamination and higher mixture ratio values indicate greater proportions of contaminating fluid relative to the physiologic patient specimen.

The mixture ratio estimator module 324 may utilize regression models trained on simulated contamination data to estimate the mixture ratio for each complete blood count trio predicted as contaminated. The regression models may be trained using the same simulated contamination data used to train the classification models, where contamination is simulated by applying linear dilution to analyte values according to a distribution of mixture ratios. The mixture ratio estimator module 324 may receive contamination predictions from the contamination predictor 318 and estimate the mixture ratio for complete blood count trios that exceed a contamination prediction threshold.

In some cases, the mixture ratio estimator module 324 may estimate mixture ratios ranging from approximately 0.05 to approximately 0.66. The distribution of estimated mixture ratios may demonstrate that contamination events with lower mixture ratios occur more frequently than contamination events with higher mixture ratios. In some cases, a median mixture ratio of approximately 0.13 to 0.14 may be observed across contaminated complete blood count trios.

The estimated mixture ratio generated by the mixture ratio estimator module 324 may be used to assess the degree to which measured analyte values in the current sample deviate from the patient's physiologic baseline due to intravenous fluid contamination. In some cases, complete blood count trios with mixture ratios above a threshold value, such as 0.10, may be selected for further analysis or validation.

The BMP validator module 326 provides orthogonal validation of contamination predictions by matching complete blood count trios predicted as contaminated with corresponding basic metabolic panels collected within a predetermined time window of the current sample. The BMP validator module 326 receives contamination predictions from the contamination predictor module 318 and identifies basic metabolic panels that were collected concurrently with contaminated complete blood count specimens.

In some cases, the BMP validator module 326 may match complete blood count trios predicted as contaminated with basic metabolic panels collected within 5 minutes of the current complete blood count sample. The 5-minute time window may be selected to identify basic metabolic panels that were collected concurrently with the complete blood count and are therefore likely to have been exposed to the same contamination event. Given that basic metabolic panels may be drawn prior to complete blood counts during specimen collection, contamination events may be more pronounced in basic metabolic panels than in paired complete blood counts when both specimens are affected by the same contaminating intravenous fluid.

The BMP validator module 326 may estimate a mixture ratio for the corresponding basic metabolic panel to provide orthogonal validation of the contamination prediction. Mixture ratios may be estimated in the corresponding basic metabolic panels using BMP-specific models trained to detect contamination in chemistry results. The BMP validator module 326 may compare the estimated mixture ratios from paired complete blood counts and basic metabolic panels to evaluate whether contamination predictions in complete blood counts are supported by evidence of contamination in concurrently collected basic metabolic panels.

In some cases, a positive correlation may be observed between the complete blood count mixture ratio and the basic metabolic panel mixture ratio for paired specimens predicted as contaminated. The correlation between contamination severity in paired complete blood count and basic metabolic panel samples provides orthogonal evidence supporting the contamination predictions generated by the machine learning model. In some cases, contamination severity in basic metabolic panels may be slightly higher on average than in corresponding complete blood counts, reflecting that basic metabolic panels are drawn prior to complete blood counts during specimen collection.

The BMP validator module 326 may generate validation outputs indicating whether contamination predictions in complete blood count trios are supported by evidence of contamination in concurrently collected basic metabolic panels. In some cases, the BMP validator module 326 may identify that a majority of basic metabolic panels collected concurrently with contaminated complete blood counts also demonstrate evidence of contamination, supporting the validity of the contamination predictions.

### Principal Component Analysis

As noted, principal component analysis (PCA) may be performed on input features to generate one or more principal components for use in detecting intravenous fluid contamination in complete blood count results. The input features for principal component analysis may comprise a hemoglobin concentration value, a platelet count value, a white blood cell count value, and delta values representing changes in analyte values between samples in a complete blood count trio. In some cases, the input features may comprise a second (current) hemoglobin concentration value, a second (current) platelet count value, and a second (current) white blood cell count value from a current sample, a prior delta comprising differences between analyte values of a prior sample and the current sample, and a post delta comprising differences between analyte values of a post sample and the current sample.

The prior delta (Δₚᵣᵢₒᵣ) may comprise a difference between a first hemoglobin concentration value from the prior sample and the second hemoglobin concentration value from the current sample, a difference between a first platelet count value from the prior sample and the second platelet count value from the current sample, and a difference between a first white blood cell count value from the prior sample and the second white blood cell count value from the current sample. The post delta (Δₚₒₛₜ) may comprise a difference between a third hemoglobin concentration value from the post sample and the second hemoglobin concentration value from the current sample, a difference between a third platelet count value from the post sample and the second platelet count value from the current sample, and a difference between a third white blood cell count value from the post sample and the second white blood cell count value from the current sample.

Given that the error-generating process of dilution by contaminating fluid is linear and proportional across all analytes, principal component analysis may effectively capture the resulting anomaly-with-resolution pattern from diluted specimens. Principal component analysis reduces the dimensionality of a complex dataset and may capture a multi-dimensional pattern in a dimensionally reduced space. In some cases, the first principal component (PC1) and the second principal component (PC2) may represent two axes that describe the anomaly-with-resolution pattern indicative of intravenous fluid contamination.

The machine learning model may receive both raw feature values and the one or more principal components as input features. In some cases, the one or more principal components may be provided as additional input features to the machine learning model along with the raw feature values comprising the hemoglobin concentration value, the platelet count value, the white blood cell count value, the prior delta, and the post delta.

Residual biological and analytical variation may remain after the variance in the dilutional effect is captured by principal components. As such, including both raw feature values and principal components may provide improved discriminatory performance by capturing both the dilutional pattern and residual variation. In some cases, three feature sets may be evaluated during model development: raw features alone, principal components alone, and a combined approach using both features and principal components.

The combined approach that includes both raw feature values and principal components may demonstrate improved discriminatory performance compared to using either features alone or principal components alone. In some cases, the features alone configuration may achieve an area under the precision-recall curve (auPR) of approximately 0.6, the principal components alone configuration may achieve an auPR of approximately 0.62, and the combined features plus principal components configuration may achieve an auPR of approximately 0.67. Model performance may be assessed by comparing results to validation sets, and the feature set with the highest auPR may be selected for use.

The combined approach using both raw feature values and principal components may be selected based on auPR performance. In some cases, the machine learning model using the combined approach may achieve an area under the receiver operating characteristic curve (auROC) of 0.972 at a first institution and 0.957 at a second institution on real-world validation sets. The machine learning model using the combined approach may achieve an auPR of 0.723 at the first institution and 0.619 at the second institution on real-world validation sets.

The machine learning model may receive input features comprising a hemoglobin concentration value, a platelet count value, and a white blood cell count value from the current sample. The input features may further comprise a prior delta comprising differences between analyte values of the prior sample and the current sample, and a post delta comprising differences between analyte values of the post sample and the current sample.

The hemoglobin concentration value may be expressed in grams per deciliter (g/dL). The platelet count value may be expressed in thousands per cubic millimeter (K/cumm). The white blood cell count value may be expressed in thousands per cubic millimeter (K/cumm). In some cases, hemoglobin concentration may be used rather than hematocrit percentage due to collinearity between hemoglobin concentration and hematocrit percentage. Given the collinearity between hemoglobin concentration and hematocrit percentage, hemoglobin concentration may be included in model development while hematocrit percentage may be excluded.

The prior delta (Δₚᵣᵢₒᵣ) may comprise a difference between a hemoglobin concentration value of the prior sample and a hemoglobin concentration value of the current sample, a difference between a platelet count value of the prior sample and a platelet count value of the current sample, and a difference between a white blood cell count value of the prior sample and a white blood cell count value of the current sample. The prior delta values capture changes in analyte concentrations from the prior sample to the current sample.

The post delta (Δₚₒₛₜ) may comprise a difference between a hemoglobin concentration value of the post sample and a hemoglobin concentration value of the current sample, a difference between a platelet count value of the post sample and a platelet count value of the current sample, and a difference between a white blood cell count value of the post sample and a white blood cell count value of the current sample. The post delta values capture changes in analyte concentrations from the current sample to the post sample.

In the anomaly-with-resolution pattern indicative of intravenous fluid contamination, the prior delta values may reflect a decrease in analyte concentrations from the prior sample to the current sample due to dilution, while the post delta values may reflect an increase in analyte concentrations from the current sample to the post sample as the measured values rebound toward the patient's baseline when a subsequent specimen is properly collected.

Delta values (Δₚᵣᵢₒᵣ, and Δₚₒₛₜ) may be calculated using the most recent result in prior and subsequent draws for each analyte within a predetermined time window. In some cases, the predetermined time window may be 48 hours. Complete blood count trios that lack prior or post measurements within the predetermined time window may be excluded from analysis.

As described above, principal component analysis may be performed on the input features to generate one or more principal components. The input features for principal component analysis may comprise the hemoglobin concentration value, the platelet count value, the white blood cell count value, and the delta values. The one or more principal components may be provided as additional input features to the machine learning model. In some cases, the machine learning model may receive both raw feature values and the one or more principal components as input features.

### Aggregation and Reporting

The systems and methods described herein may aggregate transfusion events classified as potentially unnecessary by at least one of a clinical context, a transfusion indication, a specimen collector identifier, or a collection unit identifier. Aggregation of transfusion events classified as potentially unnecessary may enable identification of patterns and trends across different dimensions of the transfusion process. In some cases, aggregation may be performed across multiple dimensions simultaneously to provide a comprehensive view of factors associated with potentially unnecessary transfusions.

Aggregation by transfusion indication may group transfusion events classified as potentially unnecessary according to the clinician-provided indication for the transfusion. Transfusion indications may include hemoglobin concentration below a threshold value, active bleeding, symptomatic anemia, and other clinical indications. In some cases, transfusions with an active bleeding indication may demonstrate lower rates of classification as potentially unnecessary compared to transfusions with other indications.

Aggregation by specimen collector identifier may group transfusion events classified as potentially unnecessary according to the individual who collected the pre-transfusion blood specimen. The specimen collector identifier may correspond to a phlebotomist, nurse, or other healthcare worker who performed the blood draw associated with the current sample in the complete blood count trio. In some cases, aggregation by specimen collector identifier may identify individuals whose collection practices result in elevated rates of potentially unnecessary transfusions.

Aggregation by collection unit identifier may group transfusion events classified as potentially unnecessary according to the hospital department or care area where the pre-transfusion blood specimen was collected. Collection units may include various hospital departments such as intensive care units, emergency departments, surgical units, and general inpatient wards. In some cases, aggregation by collection unit identifier may identify departments with elevated rates of potentially unnecessary transfusions.

The systems and methods may generate a report identifying collection units or specimen collectors associated with elevated rates of potentially unnecessary transfusions. The report may rank collection units by the proportion of transfusion events classified as potentially unnecessary relative to a total number of transfusion events within each collection unit. In some cases, the report may rank specimen collectors by the proportion of transfusion events classified as potentially unnecessary relative to a total number of transfusion events associated with specimens collected by each specimen collector.

The report may display the estimated unnecessary transfusion rate for each collection unit or specimen collector. In some cases, the report may include confidence intervals or other measures of uncertainty associated with the estimated unnecessary transfusion rates. The report may be used by laboratory personnel, transfusion medicine specialists, or quality improvement teams to focus resources on collection units or specimen collectors whose practices may benefit from additional training or process modifications.

In some cases, the report may identify collection units or specimen collectors that exceed a predetermined threshold for the rate of potentially unnecessary transfusions. The predetermined threshold may be based on institutional benchmarks, historical performance data, or comparison to peer collection units or specimen collectors. The report may prioritize collection units or specimen collectors for targeted quality improvement initiatives based on the magnitude of the deviation from the predetermined threshold.

The systems and methods may calculate an unnecessary transfusion rate based on a ratio of transfusion events classified as potentially unnecessary to a total number of transfusion events for which a complete blood count trio is available. The unnecessary transfusion rate may be expressed as a percentage representing the proportion of transfusion events that are classified as potentially unnecessary.

The denominator of the unnecessary transfusion rate may comprise the total number of transfusion events for which a complete blood count trio is available. In some cases, transfusion events that lack a matched complete blood count trio may be excluded from the calculation of the unnecessary transfusion rate. The denominator may capture the population of transfusion events at risk for misguided transfusion decisions due to intravenous fluid contamination.

In some cases, the unnecessary transfusion rate may be calculated for different subsets of transfusion events. The unnecessary transfusion rate may be calculated separately for each clinical context, transfusion indication, specimen collector identifier, or collection unit identifier. Comparison of unnecessary transfusion rates across different subsets may identify factors associated with elevated rates of potentially unnecessary transfusions.

The unnecessary transfusion rate may vary depending on the classification threshold applied to the contamination prediction. In some cases, the systems and methods may calculate the unnecessary transfusion rate at multiple classification thresholds to display the relationship between classification threshold and unnecessary transfusion rate. The analysis of unnecessary transfusion rates by classification threshold may inform selection of an appropriate threshold for a given clinical context.

As described above, the systems and methods may aggregate contamination predictions by at least one of a specimen collector identifier or a collection unit identifier to generate contamination rate data for quality improvement analysis. The contamination rate data may comprise a contamination rate for each specimen collector or collection unit, representing a proportion of complete blood count trios predicted as contaminated relative to a total number of complete blood count trios collected by that specimen collector or within that collection unit.

The contamination rate data may be used to identify specimen collectors or collection units with elevated contamination rates. In some cases, the contamination rate data may be compared to institutional benchmarks or historical performance data to identify specimen collectors or collection units that exceed expected contamination rates. The contamination rate data may support targeted quality improvement initiatives aimed at reducing intravenous fluid contamination in blood specimens.

By tracking contamination rates and unnecessary transfusion rates over time, the systems and methods may measure the effectiveness of quality improvement interventions. In some cases, a reduction in contamination rates or unnecessary transfusion rates following an intervention may indicate improvement in specimen collection practices or clinical decision-making. The aggregation and reporting capabilities may provide a mechanism for ongoing monitoring of specimen collection quality and transfusion appropriateness at institutional, departmental, and individual collector levels.

### Data Sources and Preprocessing

The systems and methods described herein may receive complete blood count data from enterprise data warehouses and laboratory information systems. In some cases, hemoglobin concentration, platelet count, and white blood cell count data may be extracted from an enterprise data warehouse at a laboratory facility. In some cases, hemoglobin concentration, platelet count, and white blood cell count data may be extracted from a laboratory information system of a hospital or healthcare facility. Given the collinearity between hemoglobin concentration and hematocrit percentage, hemoglobin concentration may be included in model development while hematocrit percentage may be excluded.

Red blood cell transfusion data may be extracted using blood product administration modules. In some cases, red blood cell transfusions may be extracted via SlicerDicer using blood product administration modules. The blood product administration modules may capture approximately 90% of all transfused units. In some cases, units administered as emergency release may not be captured by the blood product administration modules. Emergency release units may include massive transfusion protocols, whole blood, custom order sizes, and units with scanning errors. The systems and methods may exclude emergency release units from the transfusion data used for contamination prediction and unnecessary transfusion classification.

Results reported with greater than or less than symbols may be replaced with corresponding numerical values. For example, a result reported as greater than 1500 k/cumm may be replaced with a numerical value of 1500 k/cumm. In some cases, results that cannot be coerced into numerical values may be excluded from analysis. Results containing non-numerical annotations, such as comments or text-based entries, may be dropped from the dataset prior to model training and evaluation.

The prior sample and the post sample in a complete blood count trio may be collected within 48 hours of the current sample. Delta values may be calculated using the most recent result in prior and subsequent draws for each analyte within the 48-hour time window. Complete blood count trios that lack prior or post measurements within the 48-hour time window may be excluded from analysis. The 48-hour time window may be selected to capture sequential laboratory measurements that are temporally proximate to the current sample while allowing sufficient time for specimen collection in clinical workflows.

Complete blood count trios predicted as contaminated may demonstrate shorter time intervals between the current sample and the post sample compared to complete blood count trios predicted as non-contaminated. In some cases, contaminated complete blood count trios may have a median time to post sample collection of 12 to 15 hours, compared to a median time of 23 hours for non-contaminated complete blood count trios. The shorter time to post sample collection in contaminated complete blood count trios may reflect clinical workflows in which a repeat specimen is collected sooner when contamination is suspected or when anomalous results prompt additional testing.

A greater proportion of contaminated complete blood count trios may have post samples collected within shorter time intervals compared to non-contaminated complete blood count trios. In some cases, 29 to 35% of contaminated samples may have post samples collected within 8 hours of the current sample, compared to 12 to 19% for non-contaminated samples. The difference in the proportion of samples with post samples collected within 8 hours may indicate that contaminated specimens are more frequently followed by rapid repeat testing compared to non-contaminated specimens.

The systems and methods described herein may identify that contaminated complete blood count trios have shorter time to post sample collection compared to non-contaminated complete blood count trios. The identification of shorter time intervals between the current sample and the post sample in contaminated complete blood count trios may provide additional evidence supporting the contamination predictions generated by the machine learning model. In some cases, the time interval between the current sample and the post sample may be used as a characteristic associated with contamination events in quality improvement analysis.

### System Data Flow

System 300 operates through coordinated interactions among an electronic processor 304, a memory 310, and a plurality of functional modules to identify potentially unnecessary red blood cell transfusions (e.g., as part of method 100). The data flow within system 300 proceeds from CBC and/or transfusion data store(s) 308 through a series of processing stages that generate contamination predictions, evaluate hemoglobin concentrations, and classify transfusion events.

The CBC and/or transfusion data store(s) 308 provides CBC and/or transfusion data comprising complete blood count trios to the memory 310. Each complete blood count trio comprises a prior sample, a current sample, and a post sample, with each sample associated with a hemoglobin concentration value, a platelet count value, and a white blood cell count value. The electronic processor 304 retrieves the transfusion data from the memory 310 and initiates processing through the PCA module 312.

The PCA module 312 receives input features comprising the hemoglobin concentration value, the platelet count value, and the white blood cell count value from the current sample, a prior delta comprising differences between analyte values of the prior sample and the current sample, and a post delta comprising differences between analyte values of the post sample and the current sample. The PCA module 312 performs principal component analysis on the input features to generate one or more principal components. The principal components capture the anomaly-with-resolution pattern indicative of intravenous fluid contamination in a dimensionally reduced space. The PCA module 312 outputs both the raw feature values and the one or more principal components to the ML module 314, which applies the ML model 316.

The ML model 316 receives the raw feature values and the one or more principal components generated via the PCA module 312 as input features. The ML model 316 comprises a gradient boosting model tuned using cross-validation. In some cases, the ML model 316 comprises a LightGBM model tuned to log-loss in cross-validation. The ML model 316 processes the input features to generate outputs that are provided to the contamination predictor module 318 and the SHAP explainer module 328.

Contamination predictor module 318 receives outputs from the ML model 316 and generates contamination predictions for each complete blood count trio. Each contamination prediction indicates a likelihood that the current sample is contaminated by intravenous fluid. The contamination prediction may be expressed as a probability value between 0 and 1, where higher values indicate a greater likelihood of contamination. In some cases, contamination predictor 318 applies a classification threshold to the contamination prediction to generate a binary classification indicating whether the current sample is contaminated.

The contamination predictor module 318 provides contamination predictions to the hemoglobin evaluator module 320 for evaluation of transfusion events. The contamination predictor module 318 also provides contamination predictions (e.g., in parallel) to the mixture ratio estimator module 324 for estimation of contamination severity and to the BMP validator module 326 for orthogonal validation.

The hemoglobin evaluator module 320 receives contamination predictions from the contamination predictor module 318 and identifies transfusion events associated with complete blood count trios predicted as contaminated. For each transfusion event associated with a complete blood count trio predicted as contaminated, the hemoglobin evaluator module 320 evaluates whether a post-transfusion hemoglobin concentration exceeds a threshold hemoglobin value and whether the post-transfusion hemoglobin concentration exceeds a prior hemoglobin concentration. In some cases, the threshold hemoglobin value may be 8 g/dL. The hemoglobin evaluator module 320 provides evaluation results to the transfusion classifier module 322.

The transfusion classifier module 322 receives evaluation results from the hemoglobin evaluator module 320 and classifies transfusion events based on the evaluation. The transfusion classifier module 322 classifies a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration. The transfusion classifier module 322 generates classification outputs indicating whether each transfusion event is classified as potentially unnecessary or justified.

In some implementations, in parallel with the processing through the hemoglobin evaluator module 320 and the transfusion classifier module 322, the mixture ratio estimator module 324 receives contamination predictions from the contamination predictor module 318 and estimates a contamination severity for each complete blood count trio predicted as contaminated. The contamination severity comprises an estimated mixture ratio representing a proportion of contaminating fluid relative to a physiologic patient specimen. The mixture ratio estimator module 324 may utilize regression models trained on simulated contamination data to estimate the mixture ratio.

The BMP validator module 326 receives contamination predictions from the contamination predictor module 318 and provides orthogonal validation of the contamination predictions. The BMP validator module 326 matches complete blood count trios predicted as contaminated with corresponding basic metabolic panels collected within a predetermined time window of the current sample. In some cases, the predetermined time window may be 5 minutes. The BMP validator module 326 estimates a mixture ratio for the corresponding basic metabolic panel using BMP-specific models trained to detect contamination in chemistry results. The BMP validator module 326 compares the estimated mixture ratios from paired complete blood counts and basic metabolic panels to evaluate whether contamination predictions in complete blood counts are supported by evidence of contamination in concurrently collected basic metabolic panels.

The SHAP explainer module 328 receives outputs from the ML model 316 and generates feature importance values using Shapley additive explanation values. The SHAP explainer module 328 estimates each individual feature's contribution to the overall predicted probability in relation to all other feature values for each input sample. The SHAP explainer module 328 may aggregate SHAP values across all predictions to provide a global view of which features exert the strongest influence on model predictions. In some cases, the SHAP explainer module 328 may identify Principal Component 1 as having the highest impact on predictions, followed by the change in hemoglobin from current to post and the change in hemoglobin from current to prior.

The system 300 may generate summary tables using the gtsummary R package for reporting results. The summary tables may aggregate performance metrics, contamination predictions, and transfusion classifications across different dimensions such as specimen collector identifier, collection unit identifier, or clinical context. The summary tables may be used by laboratory personnel or quality improvement teams to identify patterns and trends in contamination rates and unnecessary transfusion rates.

One embodiment described herein is a method for identifying potentially unnecessary red blood cell transfusions, the method comprising: receiving, by one or more electronic processors, transfusion data comprising a plurality of red blood cell transfusion events, each transfusion event associated with a complete blood count trio comprising: a prior sample collected before a current sample; the current sample collected before the transfusion event; and a post sample collected after the transfusion event; applying, by the one or more electronic processors, a machine learning model to each complete blood count trio to generate a contamination prediction indicating a likelihood that the current sample is contaminated by intravenous fluid; for each transfusion event associated with a complete blood count trio predicted as contaminated, evaluating, by the one or more electronic processors, whether: a post-transfusion hemoglobin concentration exceeds a threshold hemoglobin value; and the post-transfusion hemoglobin concentration exceeds a prior hemoglobin concentration; and classifying, by the one or more electronic processors, a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration. In one aspect, the threshold hemoglobin value is 8 g/dL. In another aspect, the machine learning model is trained to identify an anomaly-with-resolution pattern in complete blood count results indicative of intravenous fluid contamination. In another aspect, the method further comprises: aggregating, by the one or more electronic processors, the transfusion events classified as potentially unnecessary by at least one of a clinical context, a transfusion indication, a specimen collector identifier, or a collection unit identifier. In another aspect, the method further comprises: calculating, by the one or more electronic processors, an unnecessary transfusion rate based on a ratio of transfusion events classified as potentially unnecessary to a total number of transfusion events for which a complete blood count trio is available. In another aspect, the machine learning model receives input features comprising: a plurality of analyte values comprising a hemoglobin concentration value, a platelet count value, and a white blood cell count value from each of the current sample, the prior sample, and the post sample; a prior delta for each of the plurality of analyte values comprising a difference between analyte values of the prior sample and the current sample; and a post delta for each of the plurality of analyte values comprising a difference between analyte values of the post sample and the current sample. In another aspect, the method further comprises: performing, by the one or more electronic processors, a principal component analysis on the input features to generate one or more principal components; and providing the one or more principal components as additional input features to the machine learning model. In another aspect, the method further comprises: generating, by the one or more electronic processors, a report identifying collection units or specimen collectors associated with elevated rates of potentially unnecessary transfusions. In another aspect, the prior sample and the post sample are collected within 48 hours of the current sample.

Another embodiment described herein is a system for identifying potentially unnecessary red blood cell transfusions, the system comprising: one or more electronic processors; and a memory storing instructions that, when executed by the one or more electronic processors, cause the system to: receive transfusion data comprising a plurality of red blood cell transfusion events, each transfusion event associated with a complete blood count trio comprising a prior sample collected before a current sample, the current sample collected before the transfusion event, and a post sample collected after the transfusion event; and apply a machine learning model to each complete blood count trio to generate a contamination prediction indicating a likelihood that the current sample is contaminated by intravenous fluid. In one aspect, the instructions further cause the system to: perform a principal component analysis on input features to generate one or more principal components; and provide both raw feature values and the one or more principal components as inputs to the machine learning model. In another aspect, the machine learning model is trained using simulated contamination data generated by applying linear dilution to analyte values in a training set of complete blood count trios. In another aspect, the instructions further cause the system to: estimate a mixture ratio for each complete blood count trio predicted as contaminated, the mixture ratio representing a proportion of contaminating fluid relative to a physiologic specimen. In another aspect, the instructions further cause the system to: match a complete blood count trio predicted as contaminated with a corresponding basic metabolic panel collected within a predetermined time window of the current sample; and estimate a mixture ratio for the corresponding basic metabolic panel to provide orthogonal validation of the contamination prediction. In another aspect, the instructions further cause the system to: generate feature importance values using Shapley additive explanation values to identify contributions of individual features to the contamination prediction. In another aspect, the machine learning model comprises a gradient boosting model tuned using cross-validation.

Another embodiment described herein is a non-transitory computer-readable medium storing instructions that, when executed by one or more electronic processors, cause the one or more electronic processors to: receive a plurality of complete blood count trios, each complete blood count trio comprising a prior sample, a current sample, and a post sample, each sample associated with a hemoglobin concentration value, a platelet count value, and a white blood cell count value; calculate, for each complete blood count trio, delta values representing changes in analyte values between the prior sample and the current sample and between the current sample and the post sample; apply a machine learning model trained to detect an anomaly-with-resolution pattern indicative of intravenous fluid contamination to the plurality of complete blood count trios; and generate, for each complete blood count trio, a contamination prediction indicating a likelihood that the current sample is contaminated by intravenous fluid. In one aspect, the instructions further cause the one or more electronic processors to: perform a principal component analysis on input features comprising the hemoglobin concentration value, the platelet count value, the white blood cell count value, and the delta values to generate one or more principal components; and provide both raw feature values and the one or more principal components as inputs to the machine learning model. In another aspect, the machine learning model is trained using a training set in which contamination is simulated by applying linear dilution to analyte values according to a distribution of mixture ratios. In another aspect, the instructions further cause the one or more electronic processors to: estimate a contamination severity for each complete blood count trio predicted as contaminated, the contamination severity comprising an estimated mixture ratio. In another aspect, the instructions further cause the one or more electronic processors to: identify transfusion events associated with complete blood count trios predicted as contaminated; evaluate whether a post-transfusion hemoglobin concentration exceeds a threshold value and exceeds a prior hemoglobin concentration; and classify a transfusion event as potentially unnecessary based on the evaluation. In another aspect, the instructions further cause the one or more electronic processors to: aggregate transfusion events classified as potentially unnecessary by at least one of a specimen collector identifier, a collection unit identifier, or a clinical context. In another aspect, the machine learning model comprises a gradient boosting model. In another aspect, the instructions further cause the one or more electronic processors to: apply a classification threshold to the contamination prediction to generate a binary classification indicating whether the current sample is contaminated.

Another embodiment described herein is a method for detecting intravenous fluid contamination in complete blood count results, the method comprising: receiving, by one or more electronic processors, a plurality of complete blood count trios, each complete blood count trio comprising: a prior sample associated with a first hemoglobin concentration value, a first platelet count value, and a first white blood cell count value; a current sample associated with a second hemoglobin concentration value, a second platelet count value, and a second white blood cell count value; and a post sample associated with a third hemoglobin concentration value, a third platelet count value, and a third white blood cell count value; calculating, by the one or more electronic processors, for each complete blood count trio: a prior delta comprising a difference between the first hemoglobin concentration value and the second hemoglobin concentration value, a difference between the first platelet count value and the second platelet count value, and a difference between the first white blood cell count value and the second white blood cell count value; and a post delta comprising a difference between the third hemoglobin concentration value and the second hemoglobin concentration value, a difference between the third platelet count value and the second platelet count value, and a difference between the third white blood cell count value and the second white blood cell count value; applying, by the one or more electronic processors, a machine learning model to the plurality of complete blood count trios, wherein the machine learning model is trained to distinguish contaminated samples from non-contaminated samples based on an anomaly-with-resolution pattern; and generating, by the one or more electronic processors, for each complete blood count trio, a contamination prediction indicating a likelihood that the current sample is contaminated by intravenous fluid. In one aspect, the machine learning model is trained using a training set comprising a plurality of reference complete blood count trios, wherein contamination is simulated in a portion of the plurality of reference complete blood count trios by applying a linear dilution to analyte values. In another aspect, the linear dilution is applied according to a distribution of mixture ratios representing a proportion of contaminating fluid relative to a physiologic patient specimen. In another aspect, the method further comprises: performing, by the one or more electronic processors, a principal component analysis on input features comprising the second hemoglobin concentration value, the second platelet count value, the second white blood cell count value, the prior delta, and the post delta to generate one or more principal components; and providing the one or more principal components as additional input features to the machine learning model. In another aspect, the machine learning model receives both raw feature values and the one or more principal components as input features. In another aspect, the machine learning model comprises a gradient boosting model. In another aspect, the method further comprises: applying, by the one or more electronic processors, a classification threshold to the contamination prediction to generate a binary classification indicating whether the current sample is contaminated. In another aspect, the classification threshold is selected to achieve a positive predictive value exceeding a predetermined value. In another aspect, the prior sample and the post sample are collected within 48 hours of the current sample. In another aspect, the method further comprises: estimating, by the one or more electronic processors, a contamination severity for each complete blood count trio predicted as contaminated, wherein the contamination severity comprises an estimated mixture ratio representing a proportion of contaminating fluid in the current sample. In another aspect, the method further comprises: aggregating, by the one or more electronic processors, the contamination predictions by at least one of a specimen collector identifier or a collection unit identifier to generate contamination rate data for quality improvement analysis.

Another embodiment described herein is a method for identifying potentially unnecessary red blood cell transfusions is provided. The method includes receiving, by one or more processors, transfusion data comprising a plurality of red blood cell transfusion events. Each transfusion event is associated with a complete blood count trio comprising a prior sample collected before a current sample, the current sample collected before the transfusion event, and a post sample collected after the transfusion event. The method includes applying, by the one or more processors, a machine learning model to each complete blood count trio to generate a contamination prediction indicating a likelihood that the current sample is contaminated by intravenous fluid. For each transfusion event associated with a complete blood count trio predicted as contaminated, the method includes evaluating, by the one or more processors, whether a post-transfusion hemoglobin concentration exceeds a threshold hemoglobin value and whether the post-transfusion hemoglobin concentration exceeds a prior hemoglobin concentration. The method includes classifying, by the one or more processors, a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration.

According to other aspects of the present disclosure, the method may include one or more of the following features. The threshold hemoglobin value may be 8 g/dL. The machine learning model may be trained to identify an anomaly-with-resolution pattern in complete blood count results indicative of intravenous fluid contamination. The method may further include aggregating, by the one or more processors, the transfusion events classified as potentially unnecessary by at least one of a clinical context, a transfusion indication, a specimen collector identifier, or a collection unit identifier. The method may further include calculating, by the one or more processors, an unnecessary transfusion rate based on a ratio of transfusion events classified as potentially unnecessary to a total number of transfusion events for which a complete blood count trio is available. The machine learning model may receive input features comprising a hemoglobin concentration value, a platelet count value, and a white blood cell count value from the current sample, a prior delta comprising differences between analyte values of the prior sample and the current sample, and a post delta comprising differences between analyte values of the post sample and the current sample. The method may further include performing, by the one or more processors, a principal component analysis on the input features to generate one or more principal components and providing the one or more principal components as additional input features to the machine learning model. The method may further include generating, by the one or more processors, a report identifying collection units or specimen collectors associated with elevated rates of potentially unnecessary transfusions. The prior sample and the post sample may be collected within 48 hours of the current sample.

Another embodiment described herein is a system for identifying potentially unnecessary red blood cell transfusions is provided. The system includes one or more processors and a memory storing instructions that, when executed by the one or more processors, cause the system to receive transfusion data comprising a plurality of red blood cell transfusion events. Each transfusion event is associated with a complete blood count trio comprising a prior sample collected before a current sample, the current sample collected before the transfusion event, and a post sample collected after the transfusion event. The instructions cause the system to apply a machine learning model to each complete blood count trio to generate a contamination prediction indicating a likelihood that the current sample is contaminated by intravenous fluid. For each transfusion event associated with a complete blood count trio predicted as contaminated, the instructions cause the system to evaluate whether a post-transfusion hemoglobin concentration exceeds a threshold hemoglobin value and whether the post-transfusion hemoglobin concentration exceeds a prior hemoglobin concentration. The instructions cause the system to classify a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration.

According to other aspects of the present disclosure, the system may include one or more of the following features. The instructions may further cause the system to perform a principal component analysis on input features to generate one or more principal components and provide both raw feature values and the one or more principal components as inputs to the machine learning model. The machine learning model may be trained using simulated contamination data generated by applying linear dilution to analyte values in a training set of complete blood count trios. The instructions may further cause the system to estimate a mixture ratio for each complete blood count trio predicted as contaminated, the mixture ratio representing a proportion of contaminating fluid relative to a physiologic specimen. The instructions may further cause the system to match a complete blood count trio predicted as contaminated with a corresponding basic metabolic panel collected within a predetermined time window of the current sample and estimate a mixture ratio for the corresponding basic metabolic panel to provide orthogonal validation of the contamination prediction. The instructions may further cause the system to generate feature importance values using Shapley additive explanation values to identify contributions of individual features to the contamination prediction. The machine learning model may comprise a gradient boosting model tuned using cross-validation.

Another embodiment described herein is a non-transitory computer-readable medium storing instructions is provided. The instructions, when executed by one or more processors, cause the one or more processors to receive a plurality of complete blood count trios. Each complete blood count trio comprises a prior sample, a current sample, and a post sample, each sample associated with a hemoglobin concentration value, a platelet count value, and a white blood cell count value. The instructions cause the one or more processors to calculate, for each complete blood count trio, delta values representing changes in analyte values between the prior sample and the current sample and between the current sample and the post sample. The instructions cause the one or more processors to apply a machine learning model trained to detect an anomaly-with-resolution pattern indicative of intravenous fluid contamination to the plurality of complete blood count trios. The instructions cause the one or more processors to generate, for each complete blood count trio, a contamination prediction indicating a likelihood that the current sample is contaminated by intravenous fluid.

According to other aspects of the present disclosure, the non-transitory computer-readable medium may include one or more of the following features. The instructions may further cause the one or more processors to perform a principal component analysis on input features comprising the hemoglobin concentration value, the platelet count value, the white blood cell count value, and the delta values to generate one or more principal components and provide both raw feature values and the one or more principal components as inputs to the machine learning model. The machine learning model may be trained using a training set in which contamination is simulated by applying linear dilution to analyte values according to a distribution of mixture ratios. The instructions may further cause the one or more processors to estimate a contamination severity for each complete blood count trio predicted as contaminated, the contamination severity comprising an estimated mixture ratio. The instructions may further cause the one or more processors to identify transfusion events associated with complete blood count trios predicted as contaminated, evaluate whether a post-transfusion hemoglobin concentration exceeds a threshold value and exceeds a prior hemoglobin concentration, and classify a transfusion event as potentially unnecessary based on the evaluation. The instructions may further cause the one or more processors to aggregate transfusion events classified as potentially unnecessary by at least one of a specimen collector identifier, a collection unit identifier, or a clinical context. The machine learning model may comprise a gradient boosting model. The instructions may further cause the one or more processors to apply a classification threshold to the contamination prediction to generate a binary classification indicating whether the current sample is contaminated.

Another embodiment described herein is a method for detecting intravenous fluid contamination in complete blood count results is provided. The method includes receiving, by one or more processors, a plurality of complete blood count trios. Each complete blood count trio comprises a prior sample associated with a first hemoglobin concentration value, a first platelet count value, and a first white blood cell count value, a current sample associated with a second hemoglobin concentration value, a second platelet count value, and a second white blood cell count value, and a post sample associated with a third hemoglobin concentration value, a third platelet count value, and a third white blood cell count value. The method includes calculating, by the one or more processors, for each complete blood count trio, a prior delta comprising a difference between the first hemoglobin concentration value and the second hemoglobin concentration value, a difference between the first platelet count value and the second platelet count value, and a difference between the first white blood cell count value and the second white blood cell count value. The method includes calculating a post delta comprising a difference between the third hemoglobin concentration value and the second hemoglobin concentration value, a difference between the third platelet count value and the second platelet count value, and a difference between the third white blood cell count value and the second white blood cell count value. The method includes applying, by the one or more processors, a machine learning model to the plurality of complete blood count trios, wherein the machine learning model is trained to distinguish contaminated samples from non-contaminated samples based on an anomaly-with-resolution pattern. The method includes generating, by the one or more processors, for each complete blood count trio, a contamination prediction indicating a likelihood that the current sample is contaminated by intravenous fluid.

According to other aspects of the present disclosure, the method may include one or more of the following features. The machine learning model may be trained using a training set comprising a plurality of reference complete blood count trios, wherein contamination is simulated in a portion of the plurality of reference complete blood count trios by applying a linear dilution to analyte values. The linear dilution may be applied according to a distribution of mixture ratios representing a proportion of contaminating fluid relative to a physiologic patient specimen. The method may further include performing, by the one or more processors, a principal component analysis on input features comprising the second hemoglobin concentration value, the second platelet count value, the second white blood cell count value, the prior delta, and the post delta to generate one or more principal components and providing the one or more principal components as additional input features to the machine learning model. The machine learning model may receive both raw feature values and the one or more principal components as input features. The machine learning model may comprise a gradient boosting model. The method may further include applying, by the one or more processors, a classification threshold to the contamination prediction to generate a binary classification indicating whether the current sample is contaminated. The classification threshold may be selected to achieve a positive predictive value exceeding a predetermined value. The prior sample and the post sample may be collected within 48 hours of the current sample. The method may further include estimating, by the one or more processors, a contamination severity for each complete blood count trio predicted as contaminated, wherein the contamination severity comprises an estimated mixture ratio representing a proportion of contaminating fluid in the current sample. The method may further include aggregating, by the one or more processors, the contamination predictions by at least one of a specimen collector identifier or a collection unit identifier to generate contamination rate data for quality improvement analysis.

It will be apparent to one of ordinary skill in the relevant art that suitable modifications and adaptations to the compositions, formulations, methods, processes, and applications described herein can be made without departing from the scope of any embodiments or aspects thereof. The compositions and methods provided are exemplary and are not intended to limit the scope of any of the specified embodiments. All of the various embodiments, aspects, and options disclosed herein can be combined in any variations or iterations. The scope of the compositions, formulations, methods, and processes described herein include all actual or potential combinations of embodiments, aspects, options, examples, and preferences herein described. The exemplary compositions and formulations described herein may omit any component, substitute any component disclosed herein, or include any component disclosed elsewhere herein. The ratios of the mass of any component of any of the compositions or formulations disclosed herein to the mass of any other component in the formulation or to the total mass of the other components in the formulation are hereby disclosed as if they were expressly disclosed. Should the meaning of any terms in any of the patents or publications incorporated by reference conflict with the meaning of the terms used in this disclosure, the meanings of the terms or phrases in this disclosure are controlling. Furthermore, the foregoing discussion discloses and describes merely exemplary embodiments. All patents and publications cited herein are incorporated by reference herein for the specific teachings thereof.

### Examples

### Data Collection

This study was approved by Institutional Review Boards at each institution (IRB 00185418 and 202410120). For the University of Utah (“Utah”), hemoglobin concentration (Hgb), platelet counts (Plt), and white blood cell counts (WBC) were extracted from the enterprise data warehouse at ARUP Laboratories. For Washington University School of Medicine (“WashU”), the same features were extracted from the laboratory information system of Barnes-Jewish Hospital and St. Louis Children's Hospital. Given the collinearity between hemoglobin concentration and hematocrit percentage, only hemoglobin was included in model development.

Deltas were calculated using the most recent result in prior and subsequent draws for that analyte within 48 h. If none existed, that row was removed. These complete sets of current CBC results, their changes from prior, and their changes to post will be referred to as a “CBC trio.” While no explicit patient-based inclusion/exclusion criteria were applied, this trio-based filtering likely excludes the majority of outpatient specimens. Results reported with a “>” or “<” were replaced with their corresponding numerical values (e.g., 1500 k/cumm, rather than >1500). Any result that could not be coerced into a numerical value was dropped (e.g., “See Comment”).

RBC transfusions were extracted via SlicerDicer (Epic systems, Verona, WI), using the blood product administration modules. It was estimated that around 90% of all transfused units were reliably captured with this approach, but it will not capture units given as emergency release (massive transfusion protocols, whole blood, and custom order sizes) or those with scanning errors.

### Algorithm Development

A schematic overview of the methods is provided in FIG. 1B. Machine learning (ML) pipelines were developed independently for each institution. CBC results from 2021 to 2023 were partitioned into training sets (278,447 trios at Utah, 945,736 at WashU), while results from 2024 were set aside as independent test sets (110,299 and 322,025). Contamination was simulated as a linear dilution of all analytes in a random selection of 50% of CBC trios, with all non-simulated trios labeled negative for contamination. This simulation was validated using serial dilutions of real-world patient specimens (FIG. 2). Contamination severities were drawn from a distribution of mixture ratios that mimicked real-world contamination events (FIG. 3).

The machine learning (ML) pipeline was developed using R 4.3.2 and the {tidymodels}ecosystem. Input features included the current Hgb, Plt, and WBC, as well as their relative changes when compared to their most recent prior and subsequent results for each patient. Given that the true error-generating process (dilution by contaminating fluid) is linear and proportional across all analytes, it was hypothesized that principal component analysis (PCA) would effectively capture the resulting anomaly-with-resolution pattern from diluted specimens. However, residual biological and analytical variation will remain after the variance in this dilutional effect is captured by principal components. As such, the raw feature values in addition to the principal components were included, with short tree depths and high splitting fractions to add robustness to overfitting from the simulated training set. To test these hypotheses, three feature sets were evaluated: features alone, principal components alone, and a combined approach. Final models were fit to the full training sets before being applied to the independent test sets.

Validating the Simulation with In Vitro Mixing Studies

Blood was drawn using sterile technique into EDTA vacutainer tubes and inverted immediately several times to mix. Blood was then aliquoted into specimen tubes with IV fluid (NS or D5-NS) at the mixture ratios of 0.1, 0.3, and 0.5. Total blood volume in each specimen tube, including the control (mixture ratio of 0), was 1 mL. CBC without differential was evaluated for each sample 15 minutes after sample preparation on a Sysmex XN-Series Automated Hematology System. To account for possible matrix effects, and to simulate a more real-life setting to account for time for specimen transport, each sample was run in duplicate at 45 minutes after sample preparation.

Identical experiments using normal saline (NS) and 5% dextrose in normal saline (D5-NS) were performed over two days, with the first experiment utilizing blood from Donor A and second utilizing blood from Donor B. Results are shown in the tables below (Tables 1-2) as Experiment 1 and Experiment 2, respectively.

The results from Experiment 1 and 2, at both 15 minutes and 45 minutes, are shown in Tables 1-2 and FIG. 2. The measured values (Measured NS and Measured D5-NS, solid lines) were plotted alongside the expected value calculated using the mixture ratios (Expected, dashed line).

TABLE 1
Experiment 1 - Validating the Simulation
with In Vitro Mixing Studies

| | NS - mixture ratios | | | D5-NS - mixture ratios | | |
|---|---|---|---|---|---|---|
| Control | 0.1 | 0.3 | 0.5 | 0.1 | 0.3 | 0.5 |

15 minutes

| | | | | | | | |
|---|---|---|---|---|---|---|---|
| WBC | 5.04 | 4.41 | 3.54 | 2.56 | 4.58 | 3.57 | 2.48 |
| RBC | 5.38 | 4.62 | 3.65 | 2.62 | 4.72 | 3.64 | 2.56 |
| HGB | 13.9 | 11.9 | 9.5 | 6.8 | 12.3 | 9.4 | 6.6 |
| HCT | 42 | 36.2 | 28.7 | 20.6 | 36.9 | 28.7 | 20.2 |
| MCV | 78.1 | 78.4 | 78.6 | 78.6 | 78.2 | 78.8 | 78.9 |
| PLT | 252 | 227 | 173 | 127 | 226 | 172 | 116 |

45 minutes

| | | | | | | | |
|---|---|---|---|---|---|---|---|
| WBC | 5.16 | 4.33 | 3.49 | 2.61 | 4.62 | 3.59 | 2.45 |
| RBC | 5.25 | 4.66 | 3.65 | 2.63 | 4.72 | 3.66 | 2.59 |
| HGB | 13.4 | 12 | 9.5 | 6.9 | 12.3 | 9.5 | 6.6 |
| HCT | 40.6 | 36.5 | 28.6 | 20.6 | 36.8 | 28.9 | 21.2 |
| MCV | 77.3 | 78.3 | 78.4 | 78.3 | 78 | 79 | 81.9 |
| PLT | 255 | 231 | 175 | 131 | 221 | 179 | 117 |

TABLE 2
Experiment 2 - Validating the Simulation with In Vitro Mixing Studies
15 minutes

| | | NS - mixture ratios | | | D5-NS - mixture ratios | | |
|---|---|---|---|---|---|---|---|
| | Control | 0.1 | 0.3 | 0.5 | 0.1 | 0.3 | 0.5 |
| WBC | 7.01 | 6.32 | 4.96 | 3.63 | 6.35 | 4.84 | 3.31 |
| RBC | 4.26 | 3.79 | 2.92 | 2.08 | 3.81 | 2.92 | 1.99 |
| HGB | 13.4 | 11.9 | 9.2 | 6.6 | 12 | 9.2 | 6.4 |
| HCT | 36.3 | 32.5 | 25.2 | 18.1 | 32.7 | 25.5 | 17.7 |
| MCV | 85.2 | 85.8 | 86.3 | 87 | 85.8 | 87.3 | 88.9 |
| PLT | 226 | 203 | 163 | 112 | 204 | 159 | 112 |
| WBC | 6.92 | 6.14 | 4.8 | 3.54 | 6.43 | 4.83 | 3.39 |
| RBC | 4.21 | 3.78 | 2.9 | 2.09 | 3.76 | 2.91 | 2 |
| HGB | 13.3 | 11.8 | 9.2 | 6.7 | 11.9 | 9.3 | 6.3 |
| HCT | 35.9 | 32.4 | 24.9 | 18 | 32.1 | 25.7 | 19.2 |
| MCV | 85.3 | 85.7 | 85.9 | 86.1 | 85.4 | 88.3 | 96 |
| PLT | 227 | 204 | 162 | 115 | 209 | 159 | 112 |

NS, normal saline; D5-NS, 5% dextrose in normal saline; WBC, white blood cells; RBC, red blood cells; HGB, hemoglobin; HCT, hematocrit; MCV, mean corpuscular volume; PLT, platelets. Units: WBC, K/cumm; RBC, M/cumm; HGB, g/dL; HCT, %; MCV, fl; PLT, K/cumm

### Performance Assessment

Two previously described expert-labeled validation sets from each institution were used to assess model performance. The first included 1000 randomly selected CBC trios, without exclusions, to provide an unbiased performance assessment. The second included 1000 trios for which a RBC transfusion was administered between the current and post CBC was collected to evaluate if the model's inferences were robust to transfusion-induced hemoglobin increases.

All performance assessments and summary figures were generated from each model's predictions on their institution's respective validation sets. A binary classification threshold was applied such that a positive predictive value of >0.80 would be achieved, which corresponded to a classification threshold of 0.75 in the model output. Performance metrics were calculated using the labels from manual chart review as the ground truth. Summary tables were generated using {gtsummary}. Global and local explanations of feature importance were estimated using Shapley additive explanation (SHAP) values.

### Evaluating CBC Contamination Prevalence Across Contexts

To evaluate contamination across various clinical and patient contexts, the validated models were applied retrospectively to one full year of CBC results from each institution. Contamination severity was evaluated by estimating the most likely mixture ratio of contaminating fluid relative to the physiologic patient specimen using a regression-based approach.

To provide orthogonal evidence supporting the predictions, CBC trios predicted as contaminated at a mixture ratio above 0.10 were matched to any corresponding basic metabolic panel (BMP) that was collected within 5 min of the current CBC. Mixture ratios were estimated in the corresponding BMPs using previously validated BMP-specific models, then compared in a scatterplot.

### Assessing Impact on RBC Transfusion Decisions

Additional evaluation is necessary to assess whether an RBC transfusion that is given after a contaminated CBC is potentially unnecessary. To differentiate medically justified transfusions from those that were potentially unnecessary (motivated by the dilutional effect of contamination), the following criteria were developed using the validation sets of 1000 transfusions, then applied to the full year of transfusion administrations:
The current CBC was predicted to be contaminated by the models;
The post-transfusion Hgb was >8 g/dL, to remove most cases for which empiric transfusion may be justified by AABB guidelines;
The post-transfusion hemoglobin was greater than both the prior and the current hemoglobin concentrations to remove cases for which hemoglobin decrease could have occurred for clinically significant reasons, such as acute blood loss.

All three criteria must be fulfilled to be predicted as a “potentially unnecessary” transfusion.

### Results

### Model Performance

The first performance evaluation on the simulated contamination in the training set using five-fold cross-validation is summarized in FIG. 4. The ML pipelines were highly capable of distinguishing simulated contamination from real-world results, demonstrating an area under the receiver operating characteristic curve (auROC) of 0.98 in the WashU set, and 0.99 in the Utah set, and an area under the precision-recall curve (auPR) of 0.99 for both sets.

Next, real-world performance was assessed using the validation sets from 1000 randomly selected CBC trios at each institution. The feature set with the best auPR was the approach that combined PCA with raw feature values (FIG. 5), so only these models are described below. Compared to the expert review labels, models demonstrated point estimate auROCs of 0.972 and 0.957 in Utah and WashU sets, respectively, with auPRs of 0.723 and 0.619 (FIG. 6). A binary classification threshold of 0.75 produced a sensitivity of 0.40, specificity of 0.995, positive predictive value (PPV) of 0.808, and negative predictive value (NPV) of 0.967 in the Utah set, and a sensitivity of 0.351, specificity of 0.997, PPV of 0.812, and NPV of 0.976 in the WashU set.

Model Performance in the setting of RBC Transfusion

Given the importance of the post hemoglobin in model predictions, more comprehensive evaluation of model performance on was conducted on a common edge case: patients who received an RBC transfusion between their current and post specimens (FIG. 7).

In CBCs that preceded a transfusion, the auROCs were 0.929 and 0.930, while the auPRs were 0.688 and 0.697 at Utah and WashU, respectively. Using the same specificity-focused threshold of 0.75, the sensitivity was 0.712, specificity was 0.922, PPV was 0.549, and NPV was 0.96 in the Utah set. For the WashU set, the sensitivity was 0.648, specificity was 0.919, PPV was 0.533, and NPV was 0.948.

### Rule Performance for Labeling a Transfusion as Potentially Unnecessary

To evaluate the feasibility of applying the general criteria for labeling transfusions as potentially unnecessary, positive predictions were compared to expert chart review. 81 of 84 transfusions administered after positive predictions in the Utah set and 77 of 81 in the WashU set were deemed potentially unnecessary by manual review, for a combined PPV of 96%. Transfusions deemed justified despite predicted contamination included scenarios in which the post-transfusion hemoglobin was still below 8 g/dL after the transfusion, there was active intraoperative or postoperative bleeding, and acute symptoms consistent with anemia were present.

### Application to a Full Year of CBC Results

The validated models were applied to the full, unlabeled dataset of all CBC trios collected in 2024 (Tables 3-5) at each institution. At Utah, of 288,204 CBCs performed, 110,299 (38%) had a prior and post result upon which a prediction could be made. At WashU, 322,205 (46%) of 691,118 CBCs were eligible for prediction.

TABLE 3
CBC Results Summary

| | Utah | | | | WashU | | | |
|---|---|---|---|---|---|---|---|---|
| | Train (+) | Train (−) | Test (+) | Test (−) | Train (+) | Train (−) | Test (+) | Test (−) |
| Character- | N = | N = | N = | N = | N = | N = | N = | N = |
| istic | 140,010 | 138,437 | 1,515 | 108,784 | 475,814 | 469,920 | 7,636 | 314,389 |

Hgb (g/dL)

| Median | 7.0 | 9.5 | 7.7 | 9.7 | 6.7 | 9.0 | 7.4 | 9.1 | |
|---|---|---|---|---|---|---|---|---|---|
| IQR | [5.8, 8.5] | [8.2, 11.6] | [6.8, 9.2] | [8.3, 11.7] | [5.6, 7.9] | [8.0, 10.6] | [6.7, 8.6] | [8.1, 10.8] | |
| <8 g/dL | 94,109 (67%) | 26,681 (19%) | 833 (55%) | 17,774 (16%) | 357,382 (75%) | 106,676 (23%) | 4,876 (64%) | 67,246 | (21%) |
| <7 g/dL | 69,371 (50%) | 4,121 (3%) | 458 (30%) | 2,495 (2%) | 268,665 (56%) | 18,860 (4%) | 2,625 (34%) | 10,271 | (3%) |

Platelets (K/cumm)

| Median | 140 | 199 | 146 | 199 | 143 | 202 | 147 | 201 |
|---|---|---|---|---|---|---|---|---|
| IQR | [83, 211] | [120, 289] | [74, 227] | [123, 287] | [84, 214] | [123, 294] | [75, 227] | [124, 290] |

WBC (K/cumm)

| Median | 6.7 | 9.4 | 9.0 | 9.4 | 6.4 | 8.9 | 7.6 | 9.0 |
|---|---|---|---|---|---|---|---|---|
| IQR | [4.4, 9.8] | [6.4, 13.3] | [4.6, 12.9] | [6.3, 13.3] | [4.2, 9.4] | [6.0, 12.9] | [4.8, 11.0] | [6.2, 12.9] |

ΔHgb (Prior)

| Median | −2.70 | −0.20 | −1.60 | −0.10 | −2.50 | −0.10 | −1.50 | −0.10 |
|---|---|---|---|---|---|---|---|---|
| IQR | [−3.90, −1.80] | [−0.70, 0.30] | [−2.20, −1.10] | [−0.60, 0.30] | [−3.60, −1.70] | [−0.60, 0.30] | [−2.10, −1.00] | [−0.50, 0.30] |

ΔPlt (Prior)

| Median | −49 | −1 | −30 | −1 | −48 | 0 | −27 | 0 |
|---|---|---|---|---|---|---|---|---|
| IQR | [−87, −22] | [−16, 15] | [−61, −10] | [−17, 15] | [−87, −21] | [−15, 15] | [−55, −8] | [−14, 15] |

ΔWBC (Prior)

| Median | −2.5 | −0.1 | −1.5 | −0.2 | −2.30 | −0.10 | −1.30 | −0.10 |
|---|---|---|---|---|---|---|---|---|
| IQR | [−4.6, −1.0] | [−1.4, 0.9] | [−3.4, −0.1] | [−1.4, 0.9] | [−4.30, −0.90] | [−1.20, 0.80] | [−3.00, −0.10] | [−1.20, 0.90] |

ΔHgb (Post)

| Median | 2.40 | −0.10 | 1.40 | −0.10 | 2.30 | −0.10 | 1.30 | −0.10 |
|---|---|---|---|---|---|---|---|---|
| IQR | [1.60, 3.50] | [−0.50, 0.30] | [1.00, 2.00] | [−0.50, 0.30] | [1.50, 3.40] | [−0.50, 0.40] | [0.90, 1.90] | [−0.50, 0.30] |

ΔPlt (Post)

| Median | 50 | 1 | 27 | 1 | 50 | 1 | 23 | 1 |
|---|---|---|---|---|---|---|---|---|
| IQR | [23, 89] | [−12, 18] | [10, 51] | [−13, 17] | [22, 90] | [−12, 17] | [6, 48] | [−13, 16] |

ΔWBC (Post)

| Median | 2.04 | −0.17 | 1.11 | −0.18 | 2.00 | −0.10 | 0.90 | −0.10 |
|---|---|---|---|---|---|---|---|---|
| IQR | [0.70, 3.91] | [−1.35, 0.78] | [0.09, 2.79] | [−1.36, 0.76] | [0.80, 3.80] | [−1.20, 0.80] | [0.00, 2.30] | [−1.20, 0.80] |

TABLE 4
CBC Contamination Predictions
Predictions from CBC Trios Drawn in 2024

| CBC Results | | | |
|---|---|---|---|
| WashU | | Utah | |
| Not Contaminated | Contaminated | Not Contaminated | Contaminated |
| N = 314,389 | N = 7,636 | N = 108,784 | N = 1,515 |
| (98%) | (2.4%) | (99%) | (1.4%) |

Hgb (g/dL)

| Median | 9.1 | | 7.4 | | 9.7 | | 7.7 | |
|---|---|---|---|---|---|---|---|---|
| IQR | 8.1-10.8 | | 6.7-8.6 | | 8.3-11.7 | | 6.8-9.2 | |
| <8 g/dL | 67,246 | (21%) | 4,876 | (64%) | 17,774 | (16%) | 833 | (55%) |
| <7 g/dL | 10,271 | (3%) | 2,625 | (34%) | 2,495 | (2%) | 458 | (30%) |

Hct (%)

| | | | | |
|---|---|---|---|---|
| Median | 28.2 | 22.9 | 30 | 24 |
| IQR | 25.0-33.1 | 20.5-26.6 | 26-36 | 21-28 |

WBC (K/cumm)

| | | | | |
|---|---|---|---|---|
| Median | 9 | 8 | 9 | 9 |
| IQR | 6-13 | 5-11 | 6-13 | 5-13 |

Platelets (K/cumm)

| | | | | |
|---|---|---|---|---|
| Median | 201 | 147 | 199 | 146 |
| IQR | 124-290 | 75-227 | 123-287 | 74-227 |

Hours to Post

| Median | 23 | | 15 | | 23 | | 12 | |
|---|---|---|---|---|---|---|---|---|
| IQR | 14-24 | | 7-24 | | 11-24 | | 6-24 | |
| <8 hours | 39,088 | (12%) | 2,225 | (29%) | 20,458 | (19%) | 527 | (35%) |
| <4 hours | 10,504 | (3%) | 927 | (12%) | 3,583 | (3%) | 187 | (12%) |

TABLE 5
Unnecessary Transfusion Summary
Predictions from CBC Trios Drawn in 2024

| Transfusions | | | |
|---|---|---|---|
| WashU | | Utah | |
| Justified | Unnecessary | Justified | Unnecessary |
| N = 26,272 | N = 1,708 | N = 7,225 | N = 407 |
| (94%) | (6.1%) | (95%) | (5.3%) |

Change in Hg (g/dL)

| | | | | |
|---|---|---|---|---|
| Median | 1.0 | 2.2 | 0.9 | 2.3 |
| IQR | 0.5-1.5 | 1.7-2.8 | 0.3-1.5 | 1.8-3.1 |
| Middle 95% | −1.6-3.1 | 1.2-5.1 | −2.3-3.3 | 1.2-6.3 |

Context, n (%)

| | | | | | | | | |
|---|---|---|---|---|---|---|---|---|
| ICU/Step-Down | 13,460 | (51%) | 779 | (46%) | 2,851 | (39%) | 177 | (43%) |
| Inpatient | 11,104 | (42%) | 785 | (46%) | 2,428 | (34%) | 145 | (36%) |
| OR/PACU | 848 | (3.2%) | 88 | (5.2%) | 1,501 | (21%) | 49 | (12%) |
| Other | 860 | (3.3%) | 56 | (3.3%) | 445 | (6.2%) | 36 | (8.8%) |

Indication, n (%)

| | | | | | | | | |
|---|---|---|---|---|---|---|---|---|
| Hemoglobin <7 g/dL | 7,846 | (30%) | 634 | (37%) | 2,762 | (38%) | 224 | (55%) |
| Hemoglobin <8 g/dL | 7,198 | (27%) | 447 | (26%) | 349 | (4.8%) | 17 | (4.2%) |
| Perioperative | 4,703 | (18%) | 218 | (13%) | 1,218 | (17%) | 43 | (11%) |
| Active Bleeding | 3,714 | (14%) | 257 | (15%) | 1,452 | (20%) | 67 | (16%) |
| Other | 2,286 | (8.7%) | 117 | (6.9%) | 1,372 | (19%) | 54 | (13%) |
| Not Provided | 525 | (2.0%) | 35 | (2.0%) | 72 | (1.0%) | 2 | (0.5%) |

*”Unnecessary” transfusions defined as those that follow a contaminated CBC, for which the post-transfusion hemoglobin is >8 g/dl and greater than the pre-contamination result.

Of CBC trios that were reported into the electronic medical record in 2024, 2208 (2.0%) and 7559 (2.3%) were predicted as contaminated at Utah and WashU, respectively. A summary of these CEO trios is shown in FIG. 8 and Table 6.

TABLE 6
CBC Contamination Predictions
Predictions from CBC Trios Drawn in 2024

| CBC Results | | | |
|---|---|---|---|
| WashU | | Utah | |
| Not Contaminated | Contaminated | Not Contaminated | Contaminated |
| N = 314,466 | N = 7,559 | N = 108,091 | N = 2,208 |
| (98%) | (2.3%) | (98%) | (2.0%) |

Hgb (g/dL)

| Median | 9.1 | | 7.4 | | 9.7 | | 7.7 | |
|---|---|---|---|---|---|---|---|---|
| IQR | 8.1-10.8 | | 6.7-8.6 | | 8.4-11.7 | | 6.8-9.2 | |
| <8 g/dL | 67,239 | (21%) | 4,883 | (65%) | 17,356 | (16%) | 1,251 | (57%) |
| <7 g/dL | 10,227 | (3%) | 2,669 | (35%) | 2,276 | (2%) | 677 | (31%) |

Hct (%)

| | | | | |
|---|---|---|---|---|
| Median | 28.2 | 22.8 | 30 | 24 |
| IQR | 25.0-33.1 | 20.4-26.5 | 26-36 | 21-28 |

WBC (K/cumm)

| | | | | |
|---|---|---|---|---|
| Median | 9 | 8 | 9 | 8 |
| IQR | 6-13 | 5-11 | 6-13 | 5-12 |

Platelets (K/cumm)

| | | | | |
|---|---|---|---|---|
| Median | 201 | 148 | 199 | 156 |
| IQR | 124-290 | 74-227 | 123-288 | 85-233 |

Hours to Post

| Median | 23 | | 15 | | 23 | | 12 | |
|---|---|---|---|---|---|---|---|---|
| IQR | 14-24 | | 7-24 | | 11-24 | | 6-24 | |
| <8 hours | 39,041 | (12%) | 2,272 | (30%) | 20,259 | (19%) | 726 | (33%) |
| <4 hours | 10,485 | (3%) | 946 | (13%) | 3,531 | (3%) | 239 | (11%) |

A total of 13,298 and 44,859 transfusions were extracted from the blood product administration modules at Utah and WashU, respectively, with 7632 (57%) and 27,980 (62%) having a matched CEO trio for a contamination prediction to be made. Of these, 686 (9%) and 1690 (6%) of transfusions for which a prediction could be made were flagged as potentially unnecessary using the retrospective rule set described and validated above.

Clinical Features Associated with Unnecessary Transfusion

CBC trios predicted as contaminated demonstrated the expected anomaly-with-resolution pattern, where dilutional decreases are followed by commensurate increases across each analyte (FIG. 9A). This pattern is accentuated in hemoglobin concentrations for the subset that received an RBC transfusion, which were more likely to drop below 7 g/dL in their current CBC than the non-transfused and non-contaminated counterparts.

The clinical contexts and clinician-provided transfusion indications associated with potentially unnecessary transfusions are shown in FIG. 91B and summarized in Table 7. Larger post-transfusion hemoglobin increments were more likely to be labeled potentially unnecessary, while transfusions administered peri- or intra-operatively were significantly less likely to be labeled unnecessary compared to those given in the critical care or inpatient wards. Similarly, transfusions for which the given indication was “Active bleeding” or was provided as free text were also less likely to be labeled as potentially unnecessary.

TABLE 7
Unnecessary Transfusion Summary
Predictions from CBC Trips Drawn in 2024

| WashU | | Utah | |
|---|---|---|---|
| Justified | Unnecessary | Justified | Unnecessary |
| N = 26,290 | N = 1,690 | N = 6,946 | N = 686 |
| (94%) | (6.0%) | (91%) | (9.0%) |

Change in Hg (g/dL)

| | | | | |
|---|---|---|---|---|
| Median | 1.0 | 2.2 | 0.9 | 2.3 |
| IQR | 0.5-1.5 | 1.7-2.8 | 0.3-1.5 | 1.8-3.1 |
| Middle 95% | −1.6-3.0 | 1.2-5.1 | −2.4-3.0 | 1.3-5.3 |

Context, n (%)

| | | | | | | | | |
|---|---|---|---|---|---|---|---|---|
| ICU/Step-Down | 13,463 | (51%) | 776 | (46%) | 2,730 | (39%) | 298 | (43%) |
| Inpatient | 11,115 | (42%) | 774 | (46%) | 2,301 | (33%) | 272 | (40%) |
| OR/PACU | 852 | (3.2%) | 84 | (5.0%) | 1,486 | (21%) | 64 | (9.3%) |
| Other | 860 | (3.3%) | 56 | (3.3%) | 429 | (6.2%) | 52 | (7.6%) |

Indication, n (%)

| | | | | | | | | |
|---|---|---|---|---|---|---|---|---|
| Hemoglobin <7 g/dL | 7,848 | (30%) | 632 | (37%) | 2,603 | (37%) | 383 | (56%) |
| Hemoglobin <8 g/dL | 7,221 | (27%) | 424 | (25%) | 333 | (4.8%) | 33 | (4.8%) |
| Perioperative | 4,694 | (18%) | 227 | (13%) | 1,198 | (17%) | 63 | (9.2%) |
| Active Bleeding | 3,722 | (14%) | 249 | (15%) | 1,408 | (20%) | 111 | (16%) |
| Other | 2,280 | (8.7%) | 123 | (7.3%) | 1,333 | (19%) | 93 | (14%) |
| Not Provided | 525 | (2.0%) | 35 | (2.1%) | 71 | (1.0%) | 3 | (0.4%) |

### Estimating Contamination Severity

Contamination severity, as estimated by the mixture ratio from regression models trained on simulated contamination, was evaluated for all CEO trios predicted to be contaminated by the classification models described above. The median mixture ratio at Utah was 0.13, while the median mixture ratio at WashU was 0.14 (FIG. 10). Notably, each of these trios was reported numerically—without an interpretive comment—into the electronic medical record as part of routine clinical care.

### Orthogonal Performance Evaluation: Basic Metabolic Panels

To evaluate these predictions with an orthogonal line of evidence, it was hypothesized that if a CBC was predicted to be contaminated, and a BMP was drawn at the same time, it is likely that the BMP would also display evidence of contamination. If true, given that BMPs should be drawn prior to CBCs, these contamination events should be more pronounced in BMPs than in their paired CBCs. Estimated mixture ratios from paired CBCs and BMPs are shown in FIG. 11.

Of the 4143 eligible pairs of CBCs demonstrating contamination with a mixture ratio of 0.10 or higher, 3410 BMPs (82%) also demonstrated evidence of contamination. In these contaminated pairs, a positive correlation was observed, with contamination severity in BMPs being slightly higher on average than their corresponding CBCs. The coefficient of determination was 0.56, while the line of best fit showed a slope of 1.08.

### Explaining Model and Case Predictions

To better interrogate how the models generate their predictions, explainability metrics were calculated using SHAP values. SHAP values estimate each individual feature's contribution to the overall predicted probability in relation to all other feature values for each input sample. These can be aggregated across all predictions to provide a more “global” view of which features exert the strongest influence on predictions (FIG. 12A) across the full, real-world test set.

Globally, the most contributory feature was Principal Component 1 (PC1), or the transformed projection of the axis of greatest variation in the simulated training data set, followed by additive contributions from the change in hemoglobin from current to post (Hgb_delta_post), then the change in hemoglobin from current to prior (Hgb_delta_prior). Larger decreases from prior and larger increases to post were correlated with higher predicted probabilities of contamination. Similarly, lower PC1 values were correlated with higher contamination probabilities, as the principal components appear to effectively capture the anomaly-with-resolution pattern typical of contamination, while higher values correspond to an opposite pattern across the analytes (FIG. 12B).

Described here is a proof-of-concept approach to retrospectively evaluate the prevalence of a common preanalytical error in CBCs through simulation-informed, supervised ML. Three key findings are demonstrated: (1) ML can effectively identify IV fluid contamination in CBC trios, even in the setting of RBC transfusion; (2) Contamination likely occurs in ~2% of inpatient CBC trios and is equally prevalent across institutions; and (3) these events may be motivating a substantial number of potentially unnecessary RBC transfusions. This study is believed to be the first to evaluate the potential for ML to provide scalable, robust, and generalizable assessment of the frequency and clinical impact of preanalytical errors in CBC results.

The study focused on transfusion decisions made in close proximity to contamination events as a retrospectively measurable proxy for clinical impact. While clinical decision-making around transfusion is complex, a definition for “potentially unnecessary transfusions” was developed as those that followed a contaminated CBC, for which the post-transfusion hemoglobin was greater than 8 g/dL, and greater than the prior. This definition aligns well with adjudications of clinically justified transfusions from expert review and applies to 2376 transfusions administered in 2024 across both institutions. These potentially unnecessary transfusions represent 6%-9% of all inpatient transfusions for which a CBC trio was available. While this denominator captures the population at risk for misguided transfusion decisions due to contamination and allows for a more “apples-to-apples” comparison across institutions, it does not capture all transfusions administered, most notably those without a CBC trio available for prediction. While observed contamination and unnecessary transfusion rates exceeded a priori expectations, several factors add confidence to these results. First, similar prevalences across institutions, despite heterogeneous patient populations and transfusion practices, add generalizability to these findings. Second, the orthogonal evidence observed in the frequency and correlation between contaminated CBCs and their paired BMP counterparts, combined with the biologically plausible explanation (BMPs are drawn first, by the same collector, likely in the same manner), adds a layer of robustness to spurious noise or clinical factors that may be specific to CBCs.

Finally, there is notable congruence between expected and observed feature importances estimated by SHAP. Although the inclusion of PCA makes the final pipeline inherently more difficult to explain than if it were excluded, by highlighting the extent to which PC1 effectively captures the anomaly-with-resolution pattern, the resulting pipeline remains adequately explainable. Given the appreciable performance impact of including both the raw features and the principal components from the simulated training set, it is believed that the benefits outweigh their concurrent explainability penalty. Taken together, all these factors support confidence in the models' predictions and the magnitude of the issue.

However, there is ample room for optimization and validation of this approach. Future directions should include real-world, silent, prospective evaluations where positive predictions are confirmed via ancillary testing. For example, if a specimen was suspected of dextrose contamination, measuring an extremely elevated glucose concentration should provide effective confirmation. Accruing this real-world, high-fidelity set of ground-truth labels would be crucial for adequate performance evaluation prior to live deployment and could even be used as a supplement to the simulation-based training labels. Additionally, the approach described here applies institution-specific models. The trade-off between performance and generalizability that a multi-institutional approach could provide is worthy of future exploration.

This simulation-based, wholly retrospective approach is the most notable limitation to the work described above. The lack of a gold-standard definition of contamination forced two suboptimal labeling approaches: the use of synthetic data to train the models, and manual chart review to measure their performance. Prior work has demonstrated the fallible nature of human-generated contamination predictions in chemistry panels. We hypothesize that the same is likely true in CBC results, given the alternative explanations for and mimickers of the anomaly-with-resolution pattern, such as fluid resuscitation and contraction. Additionally, this approach is unable to reliably capture transfusions that were administered outside of routine clinical workflows, such as unscanned or emergency release units, which we roughly estimated at ~10% of inpatient transfusions. Not only are these events missing from the statistical analysis, but they may have also impacted the model training and manual chart review upon which these results rely. Appropriately capturing these events, accounting for them in future training sets, and evaluating their real-world impact through prospective validation studies should be prioritized prior to implementation.

This retrospective, ML-driven approach could provide value to laboratories by evaluating the scope and impact of the issue at an institution, identifying high-value target areas for quality improvement projects, and measuring the realized impact of these efforts. Such projects could span the total testing process by focusing on improving education regarding proper phlebotomy technique, implementing better rules-based systems for detecting erroneous measurements, and developing appropriate safeguards against misguided treatment decisions. A future goal should be implementation of models that can detect erroneous results in real time, eliminating the possibility of downstream error cascades and patient harm. However, this approach would rely only on features available at the time a “current” CBC is analyzed. Developing and validating these real-time models without availability of post results is worth pursuing.

While several limitations and optimizations will need to be addressed prior to clinical implementation of any solution, retrospective or real-time, we believe the work described here is a crucial first step toward achieving these goals. Through a combination of ML tools and educational outreach, the extent and impact of this issue can be better characterized, and that laboratories and clinical services can collaborate to find effective solutions toward improving the quality of the care provided to patients.

1. A method for identifying potentially unnecessary red blood cell transfusions, the method comprising:
receiving, by one or more electronic processors, transfusion data comprising a plurality of red blood cell transfusion events, each transfusion event associated with a complete blood count trio comprising:
a prior sample collected before a current sample;
the current sample collected before the transfusion event; and
a post sample collected after the transfusion event;
applying, by the one or more electronic processors, a machine learning model to each complete blood count trio to generate a contamination prediction indicating a likelihood that the current sample is contaminated by intravenous fluid;
for each transfusion event associated with a complete blood count trio predicted as contaminated, evaluating, by the one or more electronic processors, whether:
a post-transfusion hemoglobin concentration exceeds a threshold hemoglobin value; and
the post-transfusion hemoglobin concentration exceeds a prior hemoglobin concentration; and
classifying, by the one or more electronic processors, a transfusion event as potentially unnecessary when the complete blood count trio is predicted as contaminated, the post-transfusion hemoglobin concentration exceeds the threshold hemoglobin value, and the post-transfusion hemoglobin concentration exceeds the prior hemoglobin concentration.

2. The method of claim 1, wherein the threshold hemoglobin value is 8 g/dL.

3. The method of claim 1, wherein the machine learning model is trained to identify an anomaly-with-resolution pattern in complete blood count results indicative of intravenous fluid contamination.

4. The method of claim 1, further comprising:
aggregating, by the one or more electronic processors, the transfusion events classified as potentially unnecessary by at least one of a clinical context, a transfusion indication, a specimen collector identifier, or a collection unit identifier.

5. The method of claim 1, further comprising:
calculating, by the one or more electronic processors, an unnecessary transfusion rate based on a ratio of transfusion events classified as potentially unnecessary to a total number of transfusion events for which a complete blood count trio is available.

6. The method of claim 1, wherein the machine learning model receives input features comprising:
a plurality of analyte values comprising a hemoglobin concentration value, a platelet count value, and a white blood cell count value from each of the current sample, the prior sample, and the post sample;
a prior delta for each of the plurality of analyte values comprising a difference between analyte values of the prior sample and the current sample; and
a post delta for each of the plurality of analyte values comprising a difference between analyte values of the post sample and the current sample.

7. The method of claim 6, further comprising:
performing, by the one or more electronic processors, a principal component analysis on the input features to generate one or more principal components; and
providing the one or more principal components as additional input features to the machine learning model.

8. The method of claim 1, further comprising:
generating, by the one or more electronic processors, a report identifying collection units or specimen collectors associated with elevated rates of potentially unnecessary transfusions.

9. The method of claim 1, wherein the prior sample and the post sample are collected within 48 hours of the current sample.

10. A system for identifying potentially unnecessary red blood cell transfusions, the system comprising:
one or more electronic processors; and
a memory storing instructions that, when executed by the one or more electronic processors, cause the system to:
receive transfusion data comprising a plurality of red blood cell transfusion events, each transfusion event associated with a complete blood count trio comprising a prior sample collected before a current sample, the current sample collected before the transfusion event, and a post sample collected after the transfusion event; and
apply a machine learning model to each complete blood count trio to generate a contamination prediction indicating a likelihood that the current sample is contaminated by intravenous fluid.

11. (canceled)

12. The system of claim 10, wherein the machine learning model is trained using simulated contamination data generated by applying linear dilution to analyte values in a training set of complete blood count trios.

13. The system of claim 10, wherein the instructions further cause the system to:
estimate a mixture ratio for each complete blood count trio predicted as contaminated, the mixture ratio representing a proportion of contaminating fluid relative to a physiologic specimen.

14. The system of claim 10, wherein the instructions further cause the system to:
match a complete blood count trio predicted as contaminated with a corresponding basic metabolic panel collected within a predetermined time window of the current sample; and
estimate a mixture ratio for the corresponding basic metabolic panel to provide orthogonal validation of the contamination prediction.

15. The system of claim 10, wherein the instructions further cause the system to:
generate feature importance values using Shapley additive explanation values to identify contributions of individual features to the contamination prediction.

16. The system of claim 10, wherein the machine learning model comprises a gradient boosting model tuned using cross-validation.

17. A non-transitory computer-readable medium storing instructions that, when executed by one or more electronic processors, cause the one or more electronic processors to:
receive a plurality of complete blood count trios, each complete blood count trio comprising a prior sample, a current sample, and a post sample, each sample associated with a hemoglobin concentration value, a platelet count value, and a white blood cell count value;
calculate, for each complete blood count trio, delta values representing changes in analyte values between the prior sample and the current sample and between the current sample and the post sample;
apply a machine learning model trained to detect an anomaly-with-resolution pattern indicative of intravenous fluid contamination to the plurality of complete blood count trios; and
generate, for each complete blood count trio, a contamination prediction indicating a likelihood that the current sample is contaminated by intravenous fluid.

18. The non-transitory computer-readable medium of claim 17, wherein the instructions further cause the one or more electronic processors to:
perform a principal component analysis on input features comprising the hemoglobin concentration value, the platelet count value, the white blood cell count value, and the delta values to generate one or more principal components; and
provide both raw feature values and the one or more principal components as inputs to the machine learning model.

19. The non-transitory computer-readable medium of claim 17, wherein the machine learning model is trained using a training set in which contamination is simulated by applying linear dilution to analyte values according to a distribution of mixture ratios.

20. The non-transitory computer-readable medium of claim 17, wherein the instructions further cause the one or more electronic processors to:
estimate a contamination severity for each complete blood count trio predicted as contaminated, the contamination severity comprising an estimated mixture ratio.

21-23. (canceled)

24. The non-transitory computer-readable medium of claim 17, wherein the instructions further cause the one or more electronic processors to:
apply a classification threshold to the contamination prediction to generate a binary classification indicating whether the current sample is contaminated.

25. A method for detecting intravenous fluid contamination in complete blood count results, the method comprising:
receiving, by one or more electronic processors, a plurality of complete blood count trios, each complete blood count trio comprising:
a prior sample associated with a first hemoglobin concentration value, a first platelet count value, and a first white blood cell count value;
a current sample associated with a second hemoglobin concentration value, a second platelet count value, and a second white blood cell count value; and
a post sample associated with a third hemoglobin concentration value, a third platelet count value, and a third white blood cell count value;
calculating, by the one or more electronic processors, for each complete blood count trio:
a prior delta comprising a difference between the first hemoglobin concentration value and the second hemoglobin concentration value, a difference between the first platelet count value and the second platelet count value, and a difference between the first white blood cell count value and the second white blood cell count value; and
a post delta comprising a difference between the third hemoglobin concentration value and the second hemoglobin concentration value, a difference between the third platelet count value and the second platelet count value, and a difference between the third white blood cell count value and the second white blood cell count value;
applying, by the one or more electronic processors, a machine learning model to the plurality of complete blood count trios, wherein the machine learning model is trained to distinguish contaminated samples from non-contaminated samples based on an anomaly-with-resolution pattern; and
generating, by the one or more electronic processors, for each complete blood count trio, a contamination prediction indicating a likelihood that the current sample is contaminated by intravenous fluid.

26-35. (canceled)
